(19) United States
(12) Patent Application Publication
(10) Pub. No.: US 2023/0264009 A1
(43) Pub. Date: Aug. 24, 2023

(54) DISINFECTING CAP KIT FOR LUER ACCESS DEVICES

(71) Applicant: SterileCare Inc., Markham (CA)

(72) Inventor: Karen Mueller, Newmarket (CA)

(21) Appl. No.: 18/112,648

(22) Filed: Feb. 22, 2023

Related U.S. Application Data
(60) Provisional application No. 63/396,052, filed on Aug. 8, 2022.
(60) Provisional application No. 63/312,516, filed on Feb. 22, 2022.
(60) Provisional application No. 63/312,628, filed on Feb. 22, 2022.
(60) Provisional application No. 63/312,624, filed on Feb. 22, 2022.

(51) Int. Cl. A61M 39/16

(52) U.S. Cl. CPC A61M 39/162

(57) ABSTRACT

Described herein is a luer port cleaning cap with a disinfectant comprising EDTA optimized for preserving the cap material by lowering the alcohol concentrations necessary to disinfect. Further described herein is a kit comprising the luer port cleaning cap and the composition.

### CROSS REFERENCE TO RELATED APPLICATIONS

This application claims priority to U.S. Provisional Application No. 63/312,516 entitled “SYNERGISTIC ACTIVITY OF TETRASODIUM EDTA, ETHANOL, AND CHLORHEXIDINE HYDROCHLORIDE AGAINST PLANKTONIC AND BIOFILM CELLS OF CLINICALLY RELEVANT PATHOGENS”, filed Feb. 22, 2022, and to U.S. Provisional Application No. 63/312,628 entitled “SYNERGISTIC ACTIVITY OF TETRASODIUM EDTA AND HEPARIN AGAINST PLANKTONIC AND BIOFILM CELLS OF CLINICALLY RELEVANT PATHOGENS”, filed Feb. 22, 2022, and to U.S. Provisional Application No. 63/396,052 entitled “MULTIPURPOSE SOLUTION FOR IMPROVED CATHETER LOCKS OR ENHANCED SAFETY OF IMPLANTABLE MEDICAL DEVICES”, filed Aug. 8, 2022, and to U.S. Provisional Application No. 63/312,624 entitled “LUER PORT CLEANING CAP SOLUTION” filed Feb. 22, 2022, the entire contents of each of which are incorporated by reference herein.

### FIELD OF THE DISCLOSURE

The present disclosure is generally related to cleaning solutions, and luer port caps for the cleaning of luer ports.

### BACKGROUND

Luer ports, luer access valves, needleless connectors, or luer activated devices (LADs) are commonly used in modern medicine, often used to connect tubing to various devices to administer medications or fluids. Standard configurations have been developed for several luer applications to allow tubing and components from different manufacturers of various devices to connect with one another.

These ports represent a significant avenue for infection risk. Reducing infection opportunities at luer ports can greatly reduce mortality and readmission rates. Connection sites must be sanitized, which is often done with an antiseptic wipe. End-site connectors, catheter hubs, needle access ports, or needless connectors are often cleaned using an antiseptic towelette that comes in a small foil packet and is commonly used throughout hospitals, clinics, and home healthcare. The towelette is a small, folded sheet of fibrous, non-woven material that contains isopropyl alcohol.

Luer caps are commonly used to cover a luer port when not in use. These may have a threaded, friction-based, or pressure connector to the luer port. Many luer caps have an antimicrobial solution in them, typically isopropyl alcohol. However, additional solutions are needed to reduce infection risk across a broader range of clinically relevant pathogens and to combat microbial resistance to traditional antimicrobial compositions.

### SUMMARY OF THE DISCLOSURE

Provided herein is a luer port cleaning cap device comprising: a luer cap body affixable to a luer port, the luer cap body including an interior cavity; and a seal enclosing a first composition within the interior cavity, wherein the seal is configured to release the first composition a luer access device (LAD) comprising a luer port when the luer cap body is secured to the luer port of the LAD, and wherein the first composition comprises EDTA having a concentration of at least about 1% (w/v). In some embodiments, the EDTA has a concentration in the first composition from about 1% (w/v) to about 10% (w/v), or from about 1% (w/v) to about 5% (w/v). In some embodiments, the seal is disposed within the interior cavity. In some additional embodiments, the luer cap body further comprises retention features.

In some embodiments, the luer cap body comprises a resilient thermoplastic material. In some aspects, the resilient thermoplastic material includes nylon, polycarbonate, polypropylene, or combinations thereof.

In some embodiments, the first composition has a pH of at least 6.5, or at least 9.5.

In some embodiments, the first composition further comprises heparin, taurolidine, a thrombolytic agent, or a combination thereof.

In some aspects, the first composition comprises heparin, and the heparin has a concentration in the first composition from about 1% (w/v) to about 8% (w/v), or from about 1% (w/v) to about 4% (w/v).

In some aspects, the first composition comprises a thrombolytic agent, and the thrombolytic agent comprises alteplase, streptokinase, reteplase, tenecteplase, urokinase, prourokinase, anistreplase, or a combination thereof. In an exemplary aspect, the thrombolytic agent comprises alteplase, urokinase, streptokinase, or a combination thereof. In some additional aspects, the thrombolytic agent has a concentration in the first composition of at least about 0.1% (w/v). In another aspect, the thrombolytic agent has a concentration in the first composition from about 0.1% (w/v) to about 1.5% (w/v).

In some aspects, the first composition comprises taurolidine, and the taurolidine has a concentration in the first composition from about 1% (w/v) to about 8% (w/v), or from about 1% (w/v) to about 4% (w/v).

In some embodiments, the first composition further comprises chlorhexidine or a pharmaceutically acceptable salt thereof. In some aspects, the chlorhexidine or a pharmaceutically acceptable salt thereof has a concentration in the first composition from about 0.5% (w/v) to about 6% (w/v). In some additional aspects, the chlorhexidine or a pharmaceutically acceptable salt thereof has a concentration in the first composition from about 0.1 µg/mL to about 100 µg/mL.

In some embodiments, the first composition further comprises ethanol. In some aspects, the ethanol has a concentration in the first composition from about 0.1% (w/v) to about 70% (w/v).

Further provided herein is a luer connection cleaning cap kit comprising: a luer cap including a first composition; and a wipe packet including a towelette and a second composition, wherein the first composition comprises EDTA at a concentration of at least about 1% (w/v), and wherein the second composition comprises EDTA at a concentration of at least about 1% (w/v). In some embodiments, the towelette comprises an absorbent material capable of absorbing the second composition.

In some embodiments, the first composition has a pH of at least 6.5, or at least 9.5.

In some embodiments, the first composition further comprises heparin, taurolidine, a thrombolytic agent, or a combination thereof.

In some aspects, the first composition comprises heparin, and the heparin has a concentration in the first composition from about 1% (w/v) to about 8% (w/v), or from about 1% (w/v) to about 4% (w/v).

In some aspects, the first composition comprises a thrombolytic agent, and the thrombolytic agent comprises alteplase, streptokinase, reteplase, tenecteplase, urokinase, prourokinase, anistreplase, or a combination thereof. In an exemplary aspect, the thrombolytic agent comprises alteplase, urokinase, streptokinase, or a combination thereof. In some additional aspects, the thrombolytic agent has a concentration in the first composition of at least about 0.1% (w/v). In another aspect, the thrombolytic agent has a concentration in the first composition from about 0.1% (w/v) to about 1.5% (w/v).

In some aspects, the first composition comprises taurolidine, and the taurolidine has a concentration in the first composition from about 1% (w/v) to about 8% (w/v), or from about 1% (w/v) to about 4% (w/v).

In some embodiments, the first composition further comprises chlorhexidine or a pharmaceutically acceptable salt thereof. In some aspects, the chlorhexidine or a pharmaceutically acceptable salt thereof has a concentration in the first composition from about 0.5% (w/v) to about 6% (w/v). In some additional aspects, the chlorhexidine or a pharmaceutically acceptable salt thereof has a concentration in the first composition from about 0.1 µg/mL to about 100 µg/mL.

In some embodiments, the first composition further comprises ethanol. In some aspects, the ethanol has a concentration in the first composition from about 0.1% (w/v) to about 70% (w/v).

In some embodiments, the second composition has a pH of at least 6.5, or at least 9.5.

In some embodiments, the second composition further comprises heparin, taurolidine, a thrombolytic agent, or a combination thereof.

In some aspects, the second composition comprises heparin, and the heparin has a concentration in the second composition from about 1% (w/v) to about 8% (w/v), or from about 1% (w/v) to about 4% (w/v).

In some aspects, the second composition comprises a thrombolytic agent, and the thrombolytic agent comprises alteplase, streptokinase, reteplase, tenecteplase, urokinase, prourokinase, anistreplase, or a combination thereof. In an exemplary aspect, the thrombolytic agent comprises alteplase, urokinase, streptokinase, or a combination thereof. In some additional aspects, the thrombolytic agent has a concentration in the second composition of at least about 0.1% (w/v). In another aspect, the thrombolytic agent has a concentration in the second composition from about 0.1% (w/v) to about 1.5% (w/v).

In some aspects, the first composition comprises taurolidine, and the taurolidine has a concentration in the second composition from about 1% (w/v) to about 8% (w/v), or from about 1% (w/v) to about 4% (w/v).

In some embodiments, the second composition further comprises chlorhexidine or a pharmaceutically acceptable salt thereof. In some aspects, the chlorhexidine or a pharmaceutically acceptable salt thereof has a concentration in the second composition from about 0.5% (w/v) to about 6% (w/v). In some additional aspects, the chlorhexidine or a pharmaceutically acceptable salt thereof has a concentration in the second composition from about 0.1 µg/mL to about 100 µg/mL.

In some embodiments, the second composition further comprises ethanol. In some aspects, the ethanol has a concentration in the second composition from about 0.1% (w/v) to about 70% (w/v).

### BRIEF DESCRIPTION OF THE FIGURES

FIG. 1 illustrates an exemplary luer connection cleaning cap kit of the present disclosure.

FIG. 2 illustrates an exemplary luer connection cleaning cap of the present disclosure.

FIGS. 3A-3B illustrate an exemplary determination of the minimum biofilm eradication concentration (MBEC) for tetrasodium EDTA against gram-positive, gram-negative, and fungal biofilms. CFU/mL were enumerated from each peg (n = 8) after biofilm growth for 48 h and following antimicrobial exposure for 24 h, where points on the graph represent the mean ± standard deviation from three independent experiments, and statistical significance is indicated as follows: *: P < 0.05; **: P < 0. 005; ***: P < 0 .0005; ****: P < 0.0001.

FIGS. 4A-4B illustrate an exemplary determination of the minimum biofilm eradication concentration (MBEC) for ethanol against gram-positive, gram-negative, and fungal biofilms CFU/ml were enumerated from each peg (n = 8) after biofilm growth for 48 h and following antimicrobial exposure for 24 h, where points on the graph represent the mean ± standard deviation from three independent experiments, and statistical significance is indicated as follows: *: P < 0.05; **: P < 0. 005; ***: P < 0 .0005; ****: P < 0.0001.

FIGS. 5A-5B illustrate an exemplary determination of the minimum biofilm eradication concentration (MBEC) for chlorhexidine HCl against gram-positive, gram-negative, and fungal biofilms. CFU/mL were enumerated from each peg (n = 8) after biofilm growth for 48 h and following antimicrobial exposure for 24 h, where points on the graph represent the mean ± standard deviation from three independent experiments, and statistical significance is indicated as follows: *: P < 0.05; **: P < 0. 005; ***: P < 0 .0005; ****: P < 0.0001.

FIGS. 6A-6C illustrate an exemplary efficacy of tetrasodium EDTA (TE), ethanol (ET), and chlorhexidine HCl (CH) against gram-positive and gram-negative bacterial biofilms in a 2-h exposure period, where each column represents different concentrations of test antimicrobials against each organism tested: (i) three dark grey columns represent treatment with the MBEC of TE (%), ET (%) and CH (µg/ mL); (ii) three light grey column s represents treatment with the FBEC of TE + ET, TE+ CH, and TE+ ET+ CH; (iii) white and hatched columns represent treatment with triple combinations of TE + ET+ CH, with hatched bar combinations showing the best killing effects, and statistical significance was determined by comparison with non-treated biofilms (black bar) and is indicated as follows: *: P < 0.05; **: P < 0. 005; ***: P < 0 .0005; ****: P < 0.0001. MBEC = minimum biofilm eradication concentration; FBEC = fractional biofilm eradication concentration.

FIG. 7 illustrates an exemplary efficacy of tetrasodium EDTA (TE), ethanol (ET), and chlorhexidine HCl (CH) against fungal biofilms in a 2-h exposure period, where each column represents different concentrations of test antimicrobials against each organism tested: (i) three dark grey columns represent treatment with the MBEC of TE (%), ET (%) and CH (µg /mL); (ii) three light grey column represents treatment with the FBEC of TE + ET, TE+ CH, and TE+ ET+ CH; (iii) white and hatched columns represent treatment with triple combinations of TE +ET+ CH, with hatched bar combinations showing the best killing effects, and statistical significance was determined by comparison with non-treated biofilms (black bar) and is indicated as follows: *: p < 0.05; **: p < 0.005. MBEC, minimum biofilm eradication concentration; FBEC, fractional biofilm eradication concentration.

### DETAILED DESCRIPTION

The present disclosure is directed to kits, devices, and compositions for cleaning luer ports. The compositions, which are described further below, are generally more effective than alcohol alone in destroying microbial biofilms.

### I. Kit

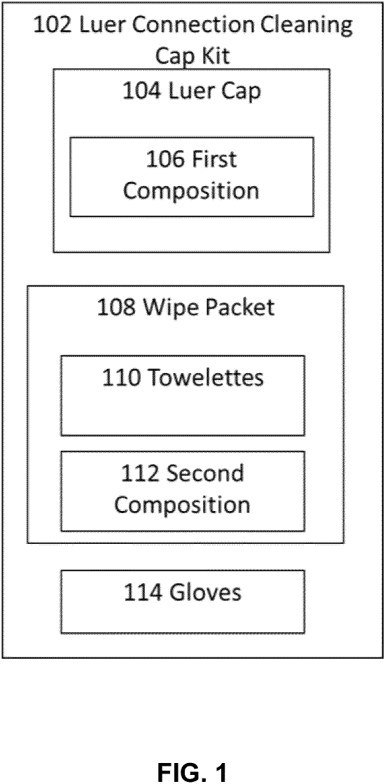

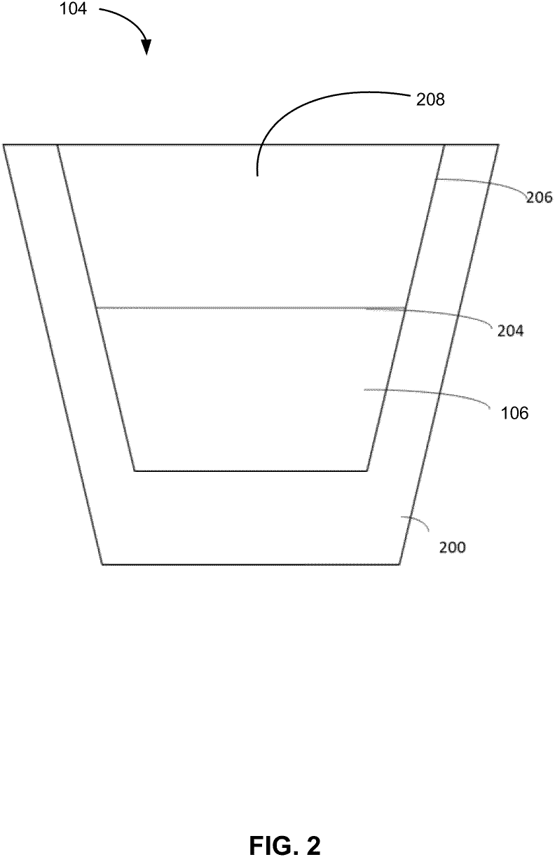

FIG. 1 illustrates an exemplary luer connection cleaning cap kit 102. The kit 102 may contain a luer cap 104. Luer caps are generally well-known to those having ordinary skill in the art. The luer cap 104 may have a threaded region and a tapered sealing region, such as an overlapping sealing region of the tapered portions of male and female luer connectors. The overlapping regions may seal a first composition 106, described in more detail below with regard to FIG. 2. The first composition 106 may be any composition described in Section II below. In some examples, the first composition 106 may be derived from the combination of tri-sodium and tetra-sodium EDTA, ethanol, and chlorhexidine at a combined tri-sodium and tetra-sodium EDTA concentration of at least 1.0% (w/v) and less than 15% (w/v), and wherein the composition has a pH of at least 9.5 and is biocompatible in a patient’s bloodstream. The first composition 106 may be adapted for specific conditions such as general maintenance of the luer cap, initial operation of the luer cap, or operation under higher risk conditions such as with an immunocompromised patient or maintenance of a luer cap that has been in use for a long period of time such as more than 1 day, 2 days, 3 days, 1 week, etc., wherein the risk of active biofilm formation is higher than normal. Thus, the first composition may have a concentration of the antimicrobial ingredients described herein adapted for the task or conditions at hand.

In some aspects, there may be two or more versions (not shown) of the first composition 106 that can be used for different conditions or tasks, such as one version of the first composition 106 that is relatively higher in taurolidine concentration and EDTA concentration than another version (not shown) of the first composition, wherein the one version with relatively higher concentrations may be used for higher-risk situations where biofilm is more likely to be present or where stronger measures against microbial infection are needed.

The luer connection cleaning cap kit 102 may also include one or more wipe packets 108. The wipe packet(s) 108 may include one or more towelettes 110 and a second composition 112. The one or more towelettes may comprise an absorbent material that absorbs the second composition 112. The absorbent material may include, for example, cotton, polyester, polypropylene, wood pulp, rayon, or other absorbent materials and combinations thereof.

The second composition 112 may be any composition described in Section II below. In some examples, the second composition 112 may be a solution derived from the combination of tri-sodium and tetra-sodium EDTA, ethanol, and chlorhexidine at a combined tri-sodium and tetra-sodium EDTA concentration of at least 1.0% (w/v) and less than 15% (w/v), and wherein the composition has a pH of at least 9.5 and is biocompatible in a patient’s bloodstream. Because the wipe packets 108 may be used for somewhat different tasks or under different conditions than the first composition 106, the second composition 112 therein may have a differing composition that may include one, two, three, or four or more of the ingredients of the first composition 106. Still, wherein one or more of the components in the second composition 112 differs from the corresponding component in the first composition 106.

By way of example, the first composition 106 may have a component such as EDTA, taurolidine, chlorhexidine, or ethanol whose concentration is at least 20% higher, 50% higher, or 100% higher (alternatively, at least 15% lower, 30% lower, or 50% lower) than the concentration of the same component in the second composition 112, or on an absolute weight percentage basis may have a concentration at least 0.5%, 1%, 2%, 5% or 8% greater (alternatively, lower by at least 0.3%, 0.5%, 1%, 2%, or 10%) than the concentration of the corresponding component in the second composition 112 or other solution provided in the kit, such as 0.5% (w/v) taurolidine in one solution and 1.5% (w/v) taurolidine in the other, for an absolute difference of 1 wt.%. Also, by way of example, the first composition 106 may have 2% (w/v) taurolidine while the second composition 112 is substantially free of taurolidine, or the first composition 106 may have from 10% (w/v) to 30% (w/v) ethanol, while the second composition 112 may have 40% (w/v) or more ethanol. Because the purpose of the first composition 106 may be primarily to prevent occlusion of the catheter or to prevent biofilm formation on or in the catheter, the first composition 106 may be adapted to be biocompatible with the physiology of the patient, while the second composition 112 in some respects need not be.

More than one type of first composition 106 or second composition 112 may be provided to cope with ongoing changes in bacteria, biofilm stage, patient physiology, condition, etc. Such multiple versions of solutions may have varying concentrations of antimicrobial or other ingredients to provide options to cope with changes over time or for different tasks.

The kit may further comprise one or more pairs of gloves 114 for a user or users to wear while handling the kit components. The gloves may be latex, nitrile, or another material suitable for medical use.

FIG. 2 illustrates one embodiment of the luer cap 104. The luer cap 104 includes a luer cap body 200 that has an opening to an interior cavity 208. The luer cap body 200 may have an inner surface and an outer surface with a coupling mechanism on the inner surface for coupling to a luer access device (LAD). LADs are generally known in the art, and are capable of connecting with luer caps.

The luer cap body 200 is affixable to a luer port. The luer cap body 200 can be made from various materials, including plastic, glass, and metal. In some embodiments, the luer cap body 200 is formed from a resilient material, such as a resilient thermoplastic material. A resilient material, as used herein, refers to a material that, when used in the intended application, deflects without permanent deformation in response to an applied force and returns to its original position when that applied force is removed. The resilient material provides for flexing or expansion of the casing in response to forces generated by the LAD when threading the cap onto the LAD device. The resiliency of the luer cap body is influenced by the type of material used to form the casing and the thickness of the casing. The resiliency may be sufficient to permit slight expansion of the casing for device threads with larger diameters while still exerting sufficient pressure on the device to create friction between the coupling mechanism and the device. In some aspects, the resilient material may include polycarbonate, polypropylene, nylon, or other thermoplastics or combinations thereof.

The coupling mechanism has retention features 206 that provide localized pressure points on the threads of the LAD to accommodate thread variance and reduce the chance a cap will prematurely disengage from the LAD. The retention features may comprise ridges, raised portions, or tapered portions of the coupling mechanism that create the localized pressure points, thus keeping the coupling mechanism in place when connected to the LAD. The coupling mechanism may further include a thread that begins proximal to the opening and spirals into the interior cavity 208. The coupling mechanism may further comprise a first lug and a second lug, wherein the first and second lugs do not intersect the thread. The first lug is closer to the opening of the casing than the second lug, and the volume of the second lug is greater than the volume of the first lug.

The interior cavity 208 may contain a first composition 106. The first composition 106 may be any composition described in Section II below or the first composition 106 described above. By way of example, the first composition 106 may be a composition derived from the combination of tri-sodium and tetra-sodium EDTA, ethanol, and chlorhexidine at a combined tri-sodium and tetra-sodium EDTA concentration of at least 1.0% (w/v) and less than 15% (w/v), and wherein the composition has a pH of at least 9.5 and is biocompatible in a patient’s bloodstream. The first composition 106 may be employed for a variety of conditions. It may include antimicrobial agents adapted for specific conditions or tasks, such as maintaining sterile conditions during the initial insertion of the catheter and for a fixed period of time after that. In contrast, a different composition may be needed for other conditions. Two or more versions (not shown) of the first composition 106 may be provided to cope with changing conditions such as changes in patient health, infection status, risk profile, environment, etc.

The first composition 106 may be held in place by a seal 204. In one embodiment, the seal 204 is affixed to the top of the luer cap body 200 by, for example, glue, solvent, or thermal bonding. The seal 204 therefore encloses the first composition 106 within the interior cavity 208. In one embodiment, such as that shown in FIG. 2, the seal 204 may be in the interior cavity of the luer cap body 200 and only broken when the luer cap body 200 is screwed onto a LAD. Thus, when the luer cap body is screwed onto a LAD, the LAD is sanitized by the first composition 106. In some embodiments, the seal may be configured for multiple caps, such as a foil strip where individual caps can be peeled from the strip as needed. These strips of caps can be made conveniently accessible by hanging them, for example, from intravenous (IV) poles or IV sets in patients’ rooms and on medication carts.

The seal 204 is capable of being broken to release the first composition from the luer cap body 200 and/or from the interior cavity 208. The seal may be broken by force (e.g. puncture), by manually peeling the seal away, by dissolution in a solvent, or by other means known in the art. In preferred embodiments, when a LAD is connected to the luer cap 104, the LAD punctures the seal 204 and releases the first composition 106, thereby sanitizing the LAD. The seal may comprise foil, plastic, or other materials.

### II. Compositions

The compositions of the present disclosure are safe for human administration and are biocompatible and non-corrosive. Preferably, the compositions are sterile. They may also have anticoagulant properties and are thus useful for preventing and/or treating a variety of catheter-related infections. The antiseptic solutions of the present disclosure have numerous applications, including applications as lock and lock flush solutions for various types of catheters, used as antiseptic agents, or solutions for sanitizing a range of medical, dental, and veterinary devices, instruments, and other objects, surfaces, and the like. They furthermore have sanitizing applications in industrial and food preparation and handling settings.

The compositions may be in the form of solutions, wherein the solvent or carrier comprises water or saline. Preferably, the composition comprises water, such as distilled, double-distilled, or deionized water.

In one embodiment, antiseptic compositions are disclosed that have at least four, and preferably at least five, of the following properties: anticoagulant properties; inhibitory and/or bactericidal activity against a broad spectrum of bacteria in a planktonic form; inhibitory and/or fungicidal activity against a spectrum of fungal pathogens; inhibitory and/or bactericidal activity against a broad spectrum of bacteria in a sessile form; inhibitory activity against protozoan infections; inhibitory activity against Acanthamoeba infections; safe and biocompatible, at least in modest volumes, in contact with a patient; safe and biocompatible, at least in modest volumes, in a patient’s bloodstream; and safe and compatible with industrial objects and surfaces.

Importantly, in most embodiments, sanitizing compositions and methods of the present disclosure do not comprise traditional antibiotic agents (e.g., beta-lactams, aminoglycosides, chloramphenicol, glycopeptides, quinolones, oxazolidinones, sulfonamides, tetracyclines, macrolides, ansamycins, streptogramins, lipopeptides, etc.) and thus do not contribute to the development of antibiotic-resistant organisms.

The compositions provided herein have activity against planktonic and biofilm cells of clinically relevant pathogens. The clinically relevant pathogens generally include, bacteria, fungi, and protists. The clinically relevant pathogens may include Gram-positive cells, Gram-negative cells, or a combination thereof. The clinically relevant pathogens include, but are not limited to, Staphylococcus (including S. epidermidis, S. aureus, and MRSA), Stenotrophomonas (including S. maltophilia), Pseudomonas (including P. Aeruginosa), Serratia (including S. marcescens), Proteus (including P. mirabilis), Escherichia (including E. coli), Klebsiella, (including K. pneumoniae), Acanthamoeba, and Candida (including C. albicans and C. parapsilosis).

The compositions provided herein comprise a salt of EDTA in solution. Sodium salts of EDTA are commonly available and may be used, including di-sodium, tri-sodium, and tetra-sodium salts, and combinations thereof. However, other EDTA salts, including ammonium, di-ammonium, potassium, di-potassium, cupric di-sodium, magnesium di-sodium, ferric sodium, and combinations thereof may also be used in addition to or instead of the sodium salts of EDTA, provided they have the antibacterial and/or fungicidal and/or anti-protozoan and/or anti-amoebic properties desired, and provided that they are sufficiently soluble in the solvent desired. In preferred embodiments, the EDTA comprises tri-sodium and tetra-sodium salts of EDTA.

The concentration of EDTA in the composition may be from about 0.5% (w/v) to about 15% (w/v), such as from about 0.5% (w/v) to about 2.5% (w/v), about 1.0% (w/v) to about 5.0% (w/v), about 1.0% (w/v) to about 7.5% (w/v), about 1.0% (w/v) to about 10% (w/v), about 1.0% (w/v) to about 12.5% (w/v), about 1.0% (w/v) to about 15% (w/v), about 2.5% (w/v) to about 15% (w/v), about 5.0% (w/v) to about 15% (w/v), about 7.5% (w/v) to about 15% (w/v), about 10% (w/v) to about 15% (w/v), about 12.5% (w/v) to about 15% (w/v), about 1% (w/v) to about 10% (w/v), or about 4% (w/v) to about 8% (w/v). Therefore, the concentration of EDTA in the composition may be about 1.0% (w/v), about 2.0% (w/v), about 3.0% (w/v), about 4.0% (w/v), about 5.0% (w/v), about 6.0% (w/v), about 7.0% (w/v), about 8.0% (w/v), about 9.0% (w/v), about 10% (w/v), about 11% (w/v), about 12% (w/v), about 13% (w/v), about 14% (w/v), or about 15% (w/v). Preferably, the EDTA has a concentration of at least about 1% (w/v).

In other embodiments, the EDTA may have a concentration in the composition from about 0.015% (w/v) to about 2% (w/v). For example, the EDTA may have a concentration in the composition from about 0.015% (w/v) to about 0.05% (w/v), about 0.015% (w/v) to about 0.1% (w/v), about 0.015% (w/v) to about 0.5% (w/v), about 0.015% (w/v) to about 1% (w/v), about 0.015% (w/v) to about 1.5% (w/v), about 0.015% (w/v) to about 2% (w/v), about 0.05% (w/v) to about 2% (w/v), about 0.1% (w/v) to about 2% (w/v), about 0.5% (w/v) to about 2% (w/v), about 1% (w/v) to about 2% (w/v), or about 1.5% (w/v) to about 2% (w/v). Therefore, the concentration of EDTA in the composition may be about 0.015% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.5% (w/v), or about 2% (w/v).

In other embodiments, the composition may be substantially free of EDTA (i.e., less than 0.001% (w/v)).

The composition may further comprise ethanol. The ethanol may be present at a concentration from about 0.1% (w/v) to about 70% (w/v). For example, the ethanol may have a concentration in the composition from about 0.1% (w/v) to about 1% (w/v), about 0.1% (w/v) to about 5% (w/v), about 0.1% (w/v) to about 10% (w/v), about 0.1% (w/v) to about 30% (w/v), about 0.1% (w/v) to about 50% (w/v), about 0.1% (w/v) to about 70% (w/v), about 1% (w/v) to about 70% (w/v), about 5% (w/v) to about 70% (w/v), about 10% (w/v) to about 70% (w/v), about 30% (w/v) to about 70% (w/v), about 50% (w/v) to about 70% (w/v), about 5% (w/v) to about 70% (w/v), about 10% (w/v) to about 50% (w/v), about 10% (w/v) to about 40% (w/v), about 10% (w/v) to about 30% (w/v), about 5% (w/v) to about 20% (w/v), or about 3.125% (w/v) to about 12.5% (w/v). Further, the composition may comprise ethanol in a concentration of about 0.1% (w/v), about 0.5% (w/v), about 1% (w/v), about 2% (w/v), about 3% (w/v), about 4% (w/v), about 5% (w/v), about 6% (w/v), about 7% (w/v), about 8% (w/v), about 9% (w/v), about 10% (w/v), about 20% (w/v), about 30% (w/v), about 40% (w/v), about 50% (w/v), about 60% (w/v), or about 70% (w/v).

In some additional embodiments, the composition may comprise a weight ratio of water to ethanol from about 10:1 to about 1:10. For example, the composition may comprise a weight ratio of water to ethanol from about 10:1 to about 8:1, about 10:1 to about 6:1, about 10:1 to about 4:1, about 10:1 to about 2:1, about 10:1 to about 1:1, about 10:1 to about 1:2, about 10:1 to about 1:4, about 10:1 to about 1:6, about 10:1 to about 1:8, about 10:1 to about 1:10, about 8:1 to about 1:10, about 6:1 to about 1:10, about 4:1 to about 1:10, about 2:1 to about 1:10, about 1:1 to about 1:10, about 1:2 to about 1:10, about 1:4 to about 1:10, about 1:6 to about 1:10, or about 1:8 to about 1:10. In additional embodiments, the composition may comprise a weight ratio of water to ethanol of about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, or about 1:10.

In other embodiments, the composition may be free of ethanol or substantially free of ethanol (i.e., less than 0.001% (w/v)).

The composition may further comprise chlorhexidine or a pharmaceutically acceptable salt thereof. Other compositions and solutions derived from chlorhexidine [1,6-bis(4′-chlorophenyl biguanide) hexane] are divalent cationic biguanide agents that exist as acetate, gluconate, and hydrochloride salts. In preferred embodiments when the composition comprises chlorhexidine, the composition comprises chlorhexidine HCl. The chlorhexidine may have a concentration in the composition from about 0.5% (w/v) to about 6% (w/v), such as from about 0.5% (w/v) to about 1% (w/v), about 0.5% (w/v) to about 2% (w/v), about 0.5% (w/v) to about 3% (w/v), about 0.5% (w/v) to about 4% (w/v), about 0.5% (w/v) to about 5% (w/v), about 0.5% (w/v) to about 6% (w/v), about 1% (w/v) to about 6% (w/v), about 2% (w/v) to about 6% (w/v), about 3% (w/v) to about 6% (w/v), about 4% (w/v) to about 6% (w/v), about 5% (w/v) to about 6%, or about 1% (w/v) to about 3% (w/v).

In another embodiment, the composition may comprise chlorhexidine in a concentration from about 0.1 µg/mL to about 100 µg/mL. For example, the composition may comprise chlorhexidine in a concentration from about 0.1 µg/mL to about 0.5 µg/mL, about 0.1 µg/mL to about 1 µg/mL, about 0.1 µg/mL to about 5 µg/mL, about 0.1 µg/mL to about 10 µg/mL, 0.1 µg/mL to about 25 µg/mL, about 0.1 µg/mL to about 50 µg/mL, about 0.1 µg/mL to about 75 µg/mL, about 0.1 µg/mL to about 100 µg/mL, about 0.5 µg/mL to about 100 µg/mL, about 1 µg/mL to about 100 µg/mL, about 5 µg/mL to about 100 µg/mL, about 10 µg/mL to about 100 µg/mL, about 25 µg/mL to about 100 µg/mL, about 50 µg/mL to about 100 µg/mL, or about 75 µg/mL to about 100 µg/mL. In some additional examples, the composition may comprise chlorhexidine at a concentration of about 0.1 µg/mL, about 0.2 µg/mL, about 0.3 µg/mL, about 0.4 µg/mL, about 0.5 µg/mL, about 0.6 µg/mL, about 0.7 µg/mL, about 0.8 µg/mL, about 0.9 µg/mL, about 1 µg/mL, about 2 µg/mL, about 3 µg/mL, about 4 µg/mL, about 5 µg/mL, about 6 µg/mL, about 7 µg/mL, about 8 µg/mL, about 9 µg/mL, about 10 µg/mL, about 20 µg/mL, about 30 µg/mL, about 40 µg/mL, about 50 µg/mL, about 60 µg/mL, about 70 µg/mL, about 80 µg/mL, about 90 µg/mL, or about 100 µg/mL. In an exemplary embodiment, composition may comprise chlorhexidine at a concentration from about 2.5 µg/mL to about 5 µg/mL, from about 0.4 µg/mL to about 50 µg/mL, or from about 0.1 µg/mL to about 50 µg/mL.

In other embodiments, the composition may be free of chlorhexidine or may be substantially free of chlorhexidine (i.e., less than 0.01 µg/mL).

The composition may further include taurolidine. The taurolidine may be present in the composition at a concentration from about 0.5% (w/v) to about 8% (w/v). For example, the taurolidine may be present at a concentration from about 0.5% (w/v) to about 1% (w/v), about 0.5% (w/v) to about 2% (w/v), about 0.5% (w/v) to about 3% (w/v), about 0.5% (w/v) to about 4% (w/v), about 0.5% (w/v) to about 5% (w/v), about 0.5% (w/v) to about 6% (w/v), about 0.5% (w/v) to about 7% (w/v), about 0.5% (w/v) to about 8% (w/v), about 1% (w/v) to about 8% (w/v), about 1.5% (w/v) to about 8% (w/v), about 2% (w/v) to about 8% (w/v), about 3% (w/v) to about 8% (w/v), about 4% (w/v) to about 8% (w/v), about 5% (w/v) to about 8% (w/v), about 6% (w/v) to about 8% (w/v), about 7% (w/v) to about 8% (w/v), about 2% (w/v) to about 7% (w/v), about 1% (w/v) to about 6% (w/v), or about 1% (w/v) to about 4% (w/v). The taurolidine may be present in the composition at a concentration of about 0.5% (w/v), about 1% (w/v), about 1.5% (w/v), about 2% (w/v), about 2.5% (w/v), about 3% (w/v), about 3.5% (w/v), about 4% (w/v), about 4.5% (w/v), about 5% (w/v), about 5.5% (w/v), about 6% (w/v), about 6.5% (w/v), about 7% (w/v), about 7.5% (w/v), or about 8% (w/v). In other embodiments, the composition may be free of taurolidine or substantially free of taurolidine (i.e., less than 0.01% w/v taurolidine).

In other embodiments, the weight ratio of EDTA to taurolidine may range from about 0.025:1 to about 40:1. For example, the weight ratio of EDTA to taurolidine may be from about 0.025:1 to about 0.1:1, about 0.025:1 to about 0.5:1, about 0.025:1 to about 1:1, about 0.025:1 to about 2:1, about 0.025:1 to about 5:1, about 0.025:1 to about 10:1, about 0.025:1 to about 25:1, about 0.025:1 to about 40:1, about 0.1:1 to about 40:1, about 0.5:1 to about 40:1, about 1:1 to about 40:1, about 2:1 to about 40:1, about 5:1 to about 40:1, about 10:1 to about 40:1, about 25:1 to about 40:1, about 0.2:1 to about 5:1, about 0.1:1 to about 1:1, about 1:1 to about 10:1, or about 0.5:1 to about 20:1. In some additional embodiments, the composition may include heparin in a weight ratio of EDTA to taurolidine of about 0.025:1, 0.05:1, 0.075:1, 0.1:1, 0.25:1, 0.5:1, 0.75:1, 1:1, 1.5:1, 2:1, 3:1, 4:1, 5:1, 10:1, 15:1, 20:1, 25:1, 30:1, 35:1, or about 40:1. In preferred embodiments, the weight ratio of EDTA to taurolidine may range from about 0.1:1 to about 10:1, about 0.2:1 to about 5:1, about 0.1:1 to about 1:1, about 1:1 to about 10:1, or about 0.5:1 to about 20:1.

The composition may further include heparin, heparan sulfate, or a combination thereof. The combination of EDTA and heparin for catheters in contact with the bloodstream of a patient has a synergistic effect in part because of the different mechanisms of EDTA and heparin relative to catheters, and in particular due to the combination of a system anti-coagulation or anti-clotting effect that heparin can induce which can reduce the attachment of blood clots to a catheter or reduce the risk of occlusion of a catheter by blood clots. In contrast, EDTA can operate via different mechanisms to hinder the formation of biofilms on the solid surfaces of the catheter and can provide an antimicrobial effect, particularly through synergy with other agents such as chlorhexidine, taurolidine, or ethanol, against planktonic and sessile bacteria. Thus, in some aspects, the benefits of EDTA combined with heparin and optionally additional antimicrobial agents may entail synergy between local antimicrobial/antibiofilm action and systemic impact on the patient.

The composition may include heparin, heparan sulfate, or a combination thereof in a concentration of at least about 0.5% (w/v). The heparin may be present in a concentration from about 1% (w/v) to about 8% (w/v), such as from about 0.5% (w/v) to about 1% (w/v), about 0.5% (w/v) to about 2% (w/v), about 0.5% (w/v) to about 3% (w/v), about 0.5% (w/v) to about 4% (w/v), about 1% (w/v) to about 5% (w/v), about 0.5% (w/v) to about 6% (w/v), about 0.5% (w/v) to about 7% (w/v), about 0.5% (w/v) to about 8% (w/v), about 2% (w/v) to about 8% (w/v), about 3% (w/v) to about 8% (w/v), about 4% (w/v) to about 8% (w/v), about 5% (w/v) to about 8% (w/v), about 6% (w/v) to about 8% (w/v), about 7% (w/v) to about 8% (w/v), about 1.5% (w/v) to about 8% (w/v), about 2% (w/v) to about 7% (w/v). In some exemplary embodiments, the heparin may be present in a concentration from about 0.5% (w/v) to about 1.8% (w/v), about 1% (w/v) to about 2.5% (w/v), or from about 0.5% (w/v) to about 4% (w/v).

The heparin, heparan sulfate, or combination thereof may further be present in a concentration of about 0.5% (w/v), about 1% (w/v), about 1.5% (w/v), about 2% (w/v), about 2.5% (w/v), about 3% (w/v), about 3.5% (w/v), about 4% (w/v), about 4.5% (w/v), about 5% (w/v), about 5.5% (w/v), about 6% (w/v), about 6.5% (w/v), about 7% (w/v), about 7.5% (w/v), or about 8% (w/v), or about 1% (w/v) to about 8% (w/v). In another embodiment, the composition may include heparin in a concentration of at least about 0.5% (w/v), at least about 1% (w/v), at least about 2% (w/v), at least about 5% (w/v), or at least about 8% (w/v). Preferably, the heparin has a concentration of at least about 1%.

In still further embodiments, the composition may be free of heparin and/or heparan sulfate or substantially free of heparin and/or heparan sulfate (i.e., less than 0.01% w/v heparin and/or heparan sulfate).

In additional embodiments, the composition may include heparin in a weight ratio of EDTA to heparin from about 0.025:1 to about 40:1. For example, the weight ratio of EDTA to heparin may be from about 0.025:1 to about 0.1:1, about 0.025:1 to about 0.5:1, about 0.025:1 to about 1:1, about 0.025:1 to about 2:1, about 0.025:1 to about 5:1, about 0.025:1 to about 10:1, about 0.025:1 to about 25:1, about 0.025:1 to about 40:1, about 0.1:1 to about 40:1, about 0.5:1 to about 40:1, about 1:1 to about 40:1, about 2:1 to about 40:1, about 5:1 to about 40:1, about 10:1 to about 40:1, about 25:1 to about 40:1, about 0.2:1 to about 5:1, about 0.1:1 to about 1:1, about 1:1 to about 10:1, or about 0.5:1 to about 20:1. In some additional embodiments, the composition may include heparin in a weight ratio of EDTA to heparin of about 0.025:1, 0.05:1, 0.075:1, 0.1:1, 0.25:1, 0.5:1, 0.75:1, 1:1, 1.5:1, 2:1, 3:1, 4:1, 5:1, 10:1, 15:1, 20:1, 25:1, 30:1, 35:1, or about 40:1.

The composition may further comprise a thrombolytic agent. Thrombolytic agents such as alteplase, urokinase, and streptokinase may be considered to deal with existing clots that cause thrombosis or occlude catheters. Such agents and their mechanisms are unrelated to EDTA’s antibacterial or anti-biofilm activity and may require different conditions than those that provide optimum performance of EDTA in a catheter lock solution. Despite multiple barriers, the Applicant has examined the possibility of an unexpected synergistic effect between EDTA and thrombolytic compounds, such that novel products and methods based on combining both classes of compounds can now be provided for improved results with implantable medical devices such as catheters. Such synergistic effects may include, but are not limited to, enhanced efficacy in preventing blood clots or undermining existing clots, enhanced efficacy in biofilm mitigation or prevention, enhanced stability or lifetime of a thrombolytic agent or solution comprising a thrombolytic agent, reduced requirement for system use of thrombolytic agents in association with implantable medical devices, etc.

The thrombolytic agent may comprise a protein or protein mixture, and more particularly may comprise an enzyme or a mixture of enzymes. In some aspects, however, the thrombolytic agent does not include heparin or aspirin. In preferred embodiments, the thrombolytic agent may comprise one or more enzymes such as alteplase, streptokinase, reteplase, tenecteplase, urokinase, prourokinase, anistreplase (APSAC), etc.

“Alteplase” is a complex fibrinolytic agent, an enzyme, that is manufactured from recombinant DNA. Sometimes it is referred to as a tissue plasminogen activator (tPA). Alteplase converts plasminogen to the proteolytic enzyme plasmin, which can lyse fibrin and fibrinogen. It is often provided commercially as a lyophilized powder in, for example, 50 mg and 100 mg vials. Each vial may be packaged with diluent (e.g., sterile water for injection) for reconstitution. It is compatible with 0.9% sodium chloride (NS) and dextrose 5% water (D5W).

“Streptokinase” is an enzyme, a purified fibrinolytic bacterial protein used to break down thrombosis in situations such as myocardial infarction, pulmonary embolism, and venous thromboembolism.

“Urokinase,” also known as urokinase-type plasminogen activator (uPA), is a serine protease present in humans and other animals. It can be described as a trypsin-like enzyme that is produced endogenously by renal parenchymal cells.

“Reteplase” may also be considered. Reteplase is a recombinant tissue plasminogen activator and modified nonglycosylated form of tPA used to dissolve intracoronary emboli, promote lysis of acute pulmonary emboli, and assist the handling of myocardial infarction. Reteplase catalyzes the cleavage of endogenous plasminogen to generate plasmin. Plasmin degrades the fibrin matrix of the thrombus. Reteplase is indicated for treating acute ST-elevation myocardial infarction (STEMI) to reduce the risk of death and heart failure.

“Prourokinase” is a relatively inactive precursor that requires the conversion to urokinase to become active.

“Tenecteplase” (TNK-tPA) is a commonly used fibrinolytic agent said to be as efficient as alteplase while exerting a lower risk of non-cerebral bleeding. Tenecteplase has higher fibrin specificity and a longer plasma half-life with final clearance, mostly through hepatic metabolism.

“Anistreplase is an anisoylated purified streptokinase activator complex (APSAC), a complex mixture of streptokinase and plasminogen that does not depend on circulating plasminogen to be effective.

Other known thrombolytic agents may be considered if they become approved for human or animal use. Such thrombolytic agents include, for example, Desmoteplase, a highly fibrin-specific thrombolytic experimental drug.

In preferred embodiments, the thrombolytic agent may comprise alteplase, urokinase, streptokinase, or a combination thereof.

The thrombolytic agent may be present in the composition at a concentration from about 0.01% (w/v) to about 1.5% (w/v). For example, the thrombolytic agent may be present in the composition at a concentration from about 0.01% to about 0.05% (w/v), about 0.01% (w/v) to about 0.1% (w/v), about 0.01% (w/v) to about 0.5% (w/v), about 0.01% (w/v) to about 1% (w/v), about 0.01% (w/v) to about 1.5% (w/v), about 0.05% (w/v) to about 1.5% (w/v), about 0.1% (w/v) to about 1.5% (w/v), about 0.5% (w/v) to about 1.5% (w/v), about 1% (w/v) to about 1.5% (w/v), or about 0.03% (w/v) to about 1.5% (w/v). In additional embodiments, the thrombolytic agent may be present in the composition at a concentration of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1% (w/v), about 1.1% (w/v), about 1.2% (w/v), about 1.3% (w/v), about 1.4% (w/v), or about 1.5% (w/v). In other embodiments, the composition may be free of a thrombolytic agent, or may be substantially free of a thrombolytic agent (i.e., less than 0.001 % (w/v)).

The composition may further comprise a surfactant, such as a cationic surfactant, an anionic surfactant, a zwitterionic surfactant, or combinations thereof. The surfactant may be a present at a concentration from about 0.5% (w/v) to about 20% (w/v). For example, the surfactant may be present at a concentration from about 0.1% (w/v) to about 5% (w/v), about 0.1% (w/v) to about 10% (w/v), about 0.1% (w/v) to about 15% (w/v), about 0.1% (w/v) to about 20% (w/v), about 5% (w/v) to about 20% (w/v), about 10% (w/v) to about 20% (w/v), about 15% (w/v) to about 20% (w/v). The surfactant may be present at a concentration of about 0.1% (w/v), about 0.5% (w/v), about 1% (w/v) about 1.5% (w/v), about 2% (w/v), about 2.5% (w/v), about 3% (w/v), about 4% (w/v), about 5% (w/v), about 6% (w/v), about 7% (w/v), about 8% (w/v), about 9% (w/v), about 10% (w/v), about 11% (w/v), about 12% (w/v), about 13% (w/v), about 14% (w/v), about 15% (w/v), about 16% (w/v), about 17% (w/v), about 18% (w/v), about 19% (w/v), or about 20% (w/v). In exemplary embodiments, the surfactant may be present at a concentration from about 0.5% (w/v) to about 20% (w/v), about 0.5% (w/v) to about 5% (w/v), about 0.1% (w/v) to about 1.5% (w/v), or less than about 2% (w/v).

The composition of the present disclosure may have a pH from about 6.5 to about 11.5. For example, the composition may have a pH from about 6.5 to about 7.5, about 6.5 to about 8.5, about 6.5 to about 9.5, about 6.5 to about 10.5, about 6.5 to about 11.5, about 7.5 to about 11.5, about 8.5 to about 11.5, about 9.5 to about 11.5, about 10.5 to about 11.5, about 6.5 to about 8, about 9.5 to about 11.5, about 9 to about 11, or about 7 to about 10. The composition may have a pH of about 6.5, 7.0, 7.5, 8.0, 8.5, 9.0, 9.5, 10.0, 10.5, 11.0, or about 11.5. In some exemplary embodiments, the composition may have a pH higher than physiological pH.

In one version, the composition may be initially at a first pH relatively closer to physiological pH, and then the pH may be increased to a pH of 8.5 or higher. Without wishing to be bound by theory, it is believed that the use of two distinct pH ranges can, in some aspects, allow a thrombolytic agent to be effective over a time period sufficiently long to act effectively against clots while at a pH relatively closer to physiological pH, while the EDTA can be more effective in its antimicrobial and/or anti-biofilm functions at the higher pH range, thereby allowing both compounds to have relatively optimum performance.

Thus in an exemplary aspect, a multipurpose solution is disclosed comprising at least one salt of ethylene diamine tetraacetic acid (EDTA) in solution, wherein at least one EDTA salt is tri-sodium or tetra-sodium EDTA, at a combined tri-sodium and tetra-sodium EDTA concentration of at least 1.0% (w/v) and less than 15% (w/v), further comprising at least 0.01% of a thrombolytic agent such as from 0.03% to 1% by weight of at least one of alteplase, urokinase, and streptokinase, wherein the multipurpose solution is obtained by combining the first solution at a first pH with a second solution at a second pH. In some aspects, the first solution comprises at least 0.03% of a thrombolytic agent, and the second solution comprises one or more salts of EDTA at a second pH. In some aspects, combining comprises the removal of a portion of the first solution followed by the addition of the second solution. In some aspects, the act of combining comprises the mixing of the first and second solutions. In some aspects, a pH control agent is added to the multipurpose solution after the first and second solutions are combined.

The composition may further comprise a buffering agent to control the pH. Suitable buffering agents for use in the composition are generally well known in the art, and may include citrate buffers, acetate buffers, and phosphate buffers.

The composition may demonstrate a broad-spectrum antimicrobial activity on a variety of planktonic and biofilm cells of clinically relevant pathogens and of sessile cells. The compositions may also eliminate a 48 hour old biofilm after a 2-hour exposure and provide a substantial reduction in biofilm cells within a 2-hour contact time.

In general, the composition is capable of eliminating greater than or equal to 75% of the strains of planktonic cells. In various embodiments, the composition may eliminate greater than or equal to 75%, greater than or equal to 80%, greater than or equal to 85%, greater than or equal to 90%, greater than or equal to 95%, or greater than or equal to 99% of the strains of planktonic cells.

Generally, the composition may eliminate greater than or equal to 75% of the strains of biofilm cells. In various embodiments, the composition eliminates greater than or equal to 75%, greater than or equal to 80%, greater than or equal top 85%, greater than or equal to 90%, greater than or equal to 95%, or greater than or equal to 99% of the strains of biofilm cells.

Preferably, the composition eliminates more than 95% of the planktonic cells. In various embodiments, the composition eliminates more than 95%, more than 96%, more than 97%, more than 98%, or more than 99% of the planktonic cells.

Preferably, the composition eliminates more than 95% of the biofilm cells. In various embodiments, the composition eliminated more than 95%, more than 96%, more than 97%, more than 98%, or more than 99% of the biofilm cells.

Preferably, the composition eliminates more than 95% of the sessile cells. In various embodiments, the composition eliminates more than 95%, more than 96%, more than 97%, more than 98%, or more than 99% of the sessile cells.

The compositions may eliminate greater than or equal to 99% biofilms following 24 hours of treatment or exposure.

Table 1 shows non-limiting exemplary compositions that may be used in the kit.

TABLE 1

| Component | First composition | Second composition |
|---|---|---|
| Carrier | 76% (w/v) | 79% (w/v) |
| Tetrasodium EDTA | 3% (w/v) | 10% (w/v) |
| Ethanol | 20% (w/v) | 10% (w/v) |
| Chlorohexidine | <1% (w/v) | <1% (w/v) |
| pH | >9.5 | >9.5 |

### III. Methods

Methods for inhibiting the growth and proliferation of microbial populations and/or fungal pathogens, including inhibiting the formation and proliferation of biofilms, are provided herein. The methods comprise contacting an infected or suspected infected object, or surface, with a composition of the present disclosure. Any composition described in Section I above may be used. Methods for inhibiting the growth and proliferation of protozoan populations are provided. The methods comprise contacting an infected or suspected infected object, or surface, with a composition of the present disclosure. Any composition described in Section I above may be used. Methods for inhibiting the growth and proliferation of amoebic populations and preventing amoebic infection, particularly Acanthamoeba infections, are also provided. The methods generally comprise contacting an object, or a surface, with a composition of the present disclosure. Any composition described in Section I above may be used.

Methods for substantially eradicating microbial populations, including both planktonic microbial populations and microbial populations in the form of biofilms, are also provided. The methods comprise contacting an infected or suspected infected object, or surface, with a composition of the present disclosure. Any composition described in Section I above may be used.

In another aspect, the method of treating an implantable medical device is provided comprising contacting a portion of the implantable medical device such as a catheter with a combination of an effective amount of a thrombolytic agent and an effective amount of one or more salts of EDTA. In a related aspect, the method may include the step of contacting the implantable medical device with the first solution at a first pH, followed by the addition of a second solution at a second pH, which yields a multipurpose solution having a pH intermediate to the first and second pH.

In another aspect, a method of preparing a catheter line for a patient is provided wherein the catheter line is provided with a lock solution that is a multipurpose solution with components and concentrations adapted for the patient’s individual needs. An automated system such as a software program or app is provided that considers data regarding the individual patient’s health and risk factors, including the potential for blood clot formation, the risk of infection in light of the patient’s immune state, age, and health, etc. Thus, for example, a patient with a history of thrombosis or adverse reactions to catheters may be provided with a lock solution with an elevated level of one or more thrombolytic agents in combination with one or more EDTA salts. In contrast, a patient with reduced risk of clot formation may be provided with a catheter lock solution with a substantially lower concentration of the thrombolytic agent. Any composition described in Section I above may be used as the catheter lock solution.

Depending on the composition used in the various methods, various compositions and contact time periods may be required to inhibit the formation and proliferation of various populations and/or to substantially eradicate various populations. Suitable contact time periods for various compositions are provided in the examples and may be determined by those having ordinary skill in the art.

### Definitions

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms have been provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the terms “a,” “an,” and “the” are understood to encompass the plural as well as the singular. Thus, the term “a mixture thereof” also relates to “mixtures thereof” and the term “a component” also refers to “components.”

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/mL” should also be understood to provide support for the range of “50 mg/mL to 80 mg/mL.”

In this disclosure, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent Law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition’s nature or characteristics would be permissible if present under the “consisting essentially of” language, even though not expressly recited in a list of items following such terminology. In this specification when using an open-ended term, like “comprising” or “including,” it is understood that direct support should be afforded also to “consisting essentially of” language as well as “consisting of” language as if stated explicitly and vice versa.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

### EXEMPLARY EMBODIMENTS

Embodiment 1: A luer port cleaning cap device comprising:
a luer cap body affixable to a luer port, the luer cap body including an interior cavity; and
a seal enclosing a first composition within the interior cavity,
wherein the seal is configured to release the first composition a luer access device (LAD) comprising a luer port when the luer cap body is secured to the luer port of the LAD, and
wherein the first composition comprises EDTA having a concentration of at least about 1% (w/v).

Embodiment 2: The device of embodiment 1, wherein the first composition has a pH of at least 6.5.

Embodiment 3: The device of embodiment 2, wherein the first composition has a pH of at least 9.5.

Embodiment 4: The device of any one of embodiments 1-3, wherein the first composition further comprises heparin, taurolidine, a thrombolytic agent, or a combination thereof.

Embodiment 5: The device of any one of embodiments 1-4, wherein the first composition further comprises chlorhexidine or a pharmaceutically acceptable salt thereof.

Embodiment 6: The device of embodiment 5, wherein the chlorhexidine or a pharmaceutically acceptable salt thereof has a concentration in the composition from about 0.5% (w/v) to about 6% (w/v).

Embodiment 7: The device of embodiment 5, wherein the chlorhexidine or a pharmaceutically acceptable salt thereof has a concentration in the composition from about 0.1 µg/mL to about 100 µg/mL.

Embodiment 8: The device of any one of embodiments 1-7, wherein the first composition further comprises ethanol.

Embodiment 9: The device of embodiment 8, wherein the ethanol has a concentration in the first composition from about 0.1% (w/v) to about 70% (w/v).

Embodiment 10: The device of any one of embodiments 1-9, wherein the EDTA has a concentration in the first composition from about 1% (w/v) to about 10% (w/v).

Embodiment 11: The device of embodiment 10, wherein the EDTA has a concentration in the first composition from about 1% (w/v) to about 5% (w/v).

Embodiment 12: The device of any one of embodiments 4-11, wherein the first composition comprises heparin, and the heparin has a concentration in the first composition from about 1% (w/v) to about 8% (w/v).

Embodiment 13: The device of embodiment 12, wherein the heparin has a concentration in the first composition from about 1% (w/v) to about 4% (w/v).

Embodiment 14: The device of any one of embodiments 4-13, wherein the first composition comprises a thrombolytic agent, and the thrombolytic agent comprises alteplase, streptokinase, reteplase, tenecteplase, urokinase, prourokinase, anistreplase, or a combination thereof.

Embodiment 15: The device of embodiment 14, wherein the thrombolytic agent comprises alteplase, urokinase, streptokinase, or a combination thereof.

Embodiment 16: The device of embodiment 14 or embodiment 15, wherein the thrombolytic agent has a concentration in the first composition of at least about 0.1% (w/v).

Embodiment 17: The device of embodiment 16, wherein the thrombolytic agent has a concentration in the first composition from about 0.1% (w/v) to about 1.5% (w/v).

Embodiment 18: The device of any one of embodiments 4-17, wherein first composition comprises taurolidine, and the taurolidine has a concentration in the first composition from about 1% (w/v) to about 8% (w/v).

Embodiment 19: The device of embodiment 18, wherein the taurolidine has a concentration in the first composition from about 1% (w/v) to about 4% (w/v).

Embodiment 20: The device of any one of embodiments 1-19, wherein the seal is disposed within the interior cavity.

Embodiment 21: The device of any one of embodiments 1-20, wherein the luer cap body further comprises retention features.

Embodiment 22: The device of any one of embodiments 1-21, wherein the luer cap body comprises a resilient thermoplastic material.

Embodiment 23: The device of embodiment 22, wherein the resilient thermoplastic material includes nylon, polycarbonate, polypropylene, or combinations thereof.

Embodiment 24: A luer connection cleaning cap kit comprising:
a luer cap including a first composition; and
a wipe packet including a towelette and a second composition,
wherein the first composition comprises EDTA at a concentration of at least about 1% (w/v), and
wherein the second composition comprises EDTA at a concentration of at least about 1% (w/v).

Embodiment 25: The kit of embodiment 24, wherein the towelette comprises an absorbent material capable of absorbing the second composition.

Embodiment 26: The kit of embodiment 24 or embodiment 25, wherein the first composition has a pH of at least 6.5.

Embodiment 27: The kit of embodiment 26, wherein the first composition has a pH of at least 9.5.

Embodiment 28: The kit of any one of embodiments 24-27, wherein the first composition further comprises heparin, taurolidine, a thrombolytic agent, or a combination thereof.

Embodiment 29: The kit of any one of embodiments 24-28, wherein the first composition further comprises chlorhexidine or a pharmaceutically acceptable salt thereof.

Embodiment 30: The kit of embodiment 29, wherein the chlorhexidine or a pharmaceutically acceptable salt thereof has a concentration in the first composition from about 0.5% (w/v) to about 6% (w/v).

Embodiment 31: The kit of embodiment 29, wherein the chlorhexidine or a pharmaceutically acceptable salt thereof has a concentration in the first composition from about 0.1 µg/mL to about 100 µg/mL.

Embodiment 32: The kit of any one of embodiments 24-31, wherein the first composition further comprises ethanol.

Embodiment 33: The kit of embodiment 32, wherein the ethanol has a concentration in the first composition from about 0.1% (w/v) to about 70% (w/v).

Embodiment 34: The kit of any one of embodiments 24-33, wherein the EDTA has a concentration in the first composition from about 1% (w/v) to about 10% (w/v).

Embodiment 35: The kit of any one of embodiments 24-34, wherein the EDTA has a concentration in the first composition from about 1% (w/v) to about 5% (w/v).

Embodiment 36: The kit of any one of embodiments 28-35, wherein the first composition comprises heparin, and the heparin has a concentration in the first composition from about 1% (w/v) to about 8% (w/v).

Embodiment 37: The kit of embodiment 36, wherein the heparin has a concentration in the first composition from about 1% (w/v) to about 4% (w/v).

Embodiment 38: The kit of any one of embodiments 28-37, wherein the first composition comprises a thrombolytic agent, and the thrombolytic agent comprises alteplase, streptokinase, reteplase, tenecteplase, urokinase, prourokinase, anistreplase, or a combination thereof.

Embodiment 39: The kit of embodiment 38, wherein the thrombolytic agent comprises alteplase, urokinase, streptokinase, or a combination thereof.

Embodiment 40: The kit of embodiment 38 or embodiment 39, wherein the thrombolytic agent has a concentration in the first composition of at least about 0.1% (w/v).

Embodiment 41: The kit of embodiment 40, wherein the thrombolytic agent has a concentration in the first composition from about 0.1% (w/v) to about 1.5% (w/v).

Embodiment 42: The kit of any one of embodiments 24-41, wherein first composition comprises taurolidine, and the taurolidine has a concentration in the first composition from about 1% (w/v) to about 8% (w/v).

Embodiment 43: The kit of embodiment 42, wherein the taurolidine has a concentration in the first composition from about 1% (w/v) to about 4% (w/v).

Embodiment 44: The kit of any one of embodiments 24-43, wherein the second composition has a pH of at least 6.5.

Embodiment 45: The kit of embodiment 44, wherein the second composition has a pH of at least 9.5.

Embodiment 46: The kit of any one of embodiments 24-45, wherein the second composition further comprises heparin, taurolidine, a thrombolytic agent, or a combination thereof.

Embodiment 47: The kit of any one of embodiments 24-46, wherein the second composition further comprises chlorhexidine or a pharmaceutically acceptable salt thereof.

Embodiment 48: The kit of embodiment 47, wherein the chlorhexidine or a pharmaceutically acceptable salt thereof has a concentration in the second composition from about 0.5% (w/v) to about 6% (w/v).

Embodiment 49: The kit of embodiment 47, wherein the chlorhexidine or a pharmaceutically acceptable salt thereof has a concentration in the second composition from about 0.1 µg/mL to about 100 µg/mL.

Embodiment 50: The kit of any one of embodiments 24-49, wherein the second composition further comprises ethanol.

Embodiment 51: The kit of embodiment 50, wherein the ethanol has a concentration in the second composition from about 0.1% (w/v) to about 70% (w/v).

Embodiment 52: The kit of any one of embodiments 24-51, wherein the EDTA has a concentration in the second composition from about 1% (w/v) to about 10% (w/v).

Embodiment 53: The kit of any one of embodiments 24-52, wherein the EDTA has a concentration in the second composition from about 1% (w/v) to about 5% (w/v).

Embodiment 54: The kit of any one of embodiments 47-53, wherein the second composition comprises heparin, and the heparin has a concentration in the second composition from about 1% (w/v) to about 8% (w/v).

Embodiment 55: The kit of embodiment 54, wherein the heparin has a concentration in the second composition from about 1% (w/v) to about 4% (w/v).

Embodiment 56: The kit of any one of embodiments 47-55, wherein the second composition comprises a thrombolytic agent, and the thrombolytic agent comprises alteplase, streptokinase, reteplase, tenecteplase, urokinase, prourokinase, anistreplase, or a combination thereof.

Embodiment 57: The kit of embodiment 56, wherein the thrombolytic agent comprises alteplase, urokinase, streptokinase, or a combination thereof.

Embodiment 58: The kit of embodiment 56 or embodiment 57, wherein the thrombolytic agent has a concentration in the second composition of at least about 0.1% (w/v).

Embodiment 59: The kit of embodiment 58, wherein the thrombolytic agent has a concentration in the second composition from about 0.1% (w/v) to about 1.5% (w/v).

Embodiment 60: The kit of any one of embodiments 24-59, wherein first composition comprises taurolidine, and the taurolidine has a concentration in the second composition from about 1% (w/v) to about 8% (w/v).

Embodiment 61: The kit of embodiment 60, wherein the taurolidine has a concentration in the second composition from about 1% (w/v) to about 4% (w/v).

### EXAMPLES

### Example 1

A organisms and culture composition included (1) test pathogens of a single isolate from the species Stenotrophomonas maltophilia (ON17), Proteus mirabilis (ON153), Pseudomonas aeruginosa (SK1), and Serratia marcescens (SI<2) as well as (2) two isolates from the species Staphylococcus epidermidis (ON170 and SK9), S. aureus (ON89 and ON184), E. coli (ON29 and SK2) and Candida albicans (ON47 and SK4b).

An antimicrobials composition included a KiteLock™ 4% Sterile Catheter Lock Solution (40 mg/mL tetrasodium EDTA) by SterileCare Inc., which is distinct from standard ‘disodium’ EDTA that is prepared at near-neutral pH; the pH of the KiteLock™ solution is near 11. The high pH does not kill micro-organisms directly but changes EDTA to the tetrasodium form, which has increased microbial killing effects. Chlorhexidine HCl was purchased from Sigma-Aldrich (product #C8527-5G). The antimicrobials composition (1 mg/mL) was made by dissolving the appropriate amount of chlorhexidine HCl powder in distilled water heated to 50° C., allowing the solution to cool and passing it through a 0.22 µm filter.

An assay was made using a minimum inhibitory concentration (MIC) and minimum bactericidal concentration (MBC) and minimum fungicidal concentration (MFC) determination. The MIC was determined by the micro broth dilution method in 96-well plates. Serial two-fold dilutions of tetrasodium EDTA (from 2% to 0.015%), ethanol (from 50% to 0.1%) and chlorhexidine HCl (from 100 µg/mL to 0.025 µg/mL) were prepared in MH broth with a final volume of 90 µL per well. A 10 µL containing 1 × 10⁵ bacterial cells or 2 × 10³ fungal cells were added to each well. The inoculated plates were covered with a lid, sealed with Parafilm, and incubated for 24 h at 37° C. with slight rocking on a tilting platform shaker. After incubation, the optical density at 600 nm (OD ₆₀₀ ) of the cultures in each well was measured using an xMark™ Microplate Absorbance Spectrophotometer (Bio-Rad). The MIC was defined as the lowest concentration of antimicrobial compound at which the culture OD₆₀₀ values were similar to uninoculated control wells. MBCs and MFCs were determined by transferring 100 µL from each well with no apparent growth onto appropriate agar plates, followed by incubation for 24 h at 37° C.

A fourth composition and solution included tetrasodium EDTA with ethanol or chlorhexidine HCl. This fourth composition and solution were created using checkerboard titration methods using micro broth dilution in 96-well microtiter plates. The concentrations of antimicrobials used were based on previously determined MIC values. Briefly, 200 µL of two-fold dilutions of tetrasodium EDTA and ethanol or chlorhexidine HCl were prepared in MH or MH II broth with standardized cell suspension. The plate contained decreasing concentrations of tetrasodium EDTA (2%- 0.015%) in columns 1-10 and decreasing concentrations of ethanol (50%-0.4%) or chlorhexidine HCl (50 µg/mL-0.0125 µg/mL) in rows A-H. Then, 10 µL of standardized cell suspension was added to each well. Microtiter plates were incubated at 37° C. for 24 h, and the results were analyzed. Each test was performed in duplicate and included a growth control without adding any antimicrobials.

A biofilm cultivation cell composition was provided. This fifth composition was created using an MBEC Assay® biofilm inoculator, consisting of a polystyrene lid with 96 downward-protruding pegs and a corresponding base used to grow biofilms. A standardized inoculum was diluted in an appropriate biofilm growth medium to achieve a viable cell count of 1.5 × 10⁶ CFU/mL of bacterial cells or 5 ×7 10⁵ CFU/mL of fungal cells. Then, 150 µL of this inoculum was transferred into each appropriate well, and the peg lids were inserted into the microtiter plates. The plates were sealed with Parafilm and were incubated at optimum temperature for 48 h with slight rocking for bacteria and shaking at 200 rpm for fungal strains. After incubation, the peg lid was removed from the base and rinsed twice with sterile phosphate-buffered saline (PBS) for 2 min to remove loosely attached non-sessile cells. Before the antimicrobial challenge, the pegs in column 1 (n = 8) were considered the biofilm growth control; these pegs were removed from the lids, placed into 200 µL of recovery medium, and analyzed for starting biofilm cell numbers as described below. The rinsed pegs were placed into new 96-well plates containing two-fold dilutions of antimicrobials such as tetrasodium EDTA (4 %-0.0125%), ethanol (100%-0.2%), and chlorhexidine HCl (100 µg/mL-0.4 µg/mL) in 200 µL of suitable biofilm growth medium per well and incubated at optimum temperature for 24 h. After the antimicrobial challenge, the pegs were rinsed twice with sterile PBS for 2 min and placed into a new 96-well plate containing 200 µL of recovery medium. The recovery plates were sealed with Parafilm, and biofilm cells were dislodged from the pegs by sonication for 30 min with a Branson 3510 bath sonicator. The biofilm cells in the recovery medium were serially diluted, and a drop dilution assay was performed to enumerate the viable cells. MBEC values were determined as the minimum concentration of antimicrobials that yielded a viable cell count at or lower than the 125 CFU/mL detection limit.

Determining the fractional biofilm eradication concentration (FBEC) index included the steps of (1) identifying synergistic antimicrobial effects of tetrasodium EDTA with either ethanol or chlorhexidine HCl on established biofilms, (2) using the ‘checkerboard dilution method’ where (3) pegs containing biofilms were treated with a combination of tetrasodium EDTA and ethanol or with tetrasodium EDTA and chlorhexidine HCl in 200 µL of two-fold dilutions inappropriate biofilm growth medium. This was followed by step (4) that included eight dilution steps of tetrasodium EDTA (4 %- 0.015%) either with ethanol (50%- 0.4%) or chlorhexidine HCl (50 µg/mL- 0.4 µg/mL) and where eight growth controls are analyzed for synergistic biofilm eradication. In step (4), microtiter plates are incubated at 37° C. for 24 h. then (6), after incubation, the bacterial and fungal cells were dislodged from the pegs into the recovery medium described above.

Three 10-µL aliquots, for a total of 30 µL from each well of recovery medium, were spotted on MH agar plates and incubated for 24 h at 37° C. The FBEC is the minimum concentration of antimicrobials in combination that completely inhibited bacterial or fungal growth on agar plates. The FBEC determination is a modification of the FICI.

Determining rapid biofilm eradication by tetrasodium EDTA, ethanol, and chlorhexidine HCl alone and in combination was performed. After biofilm formation, control pegs (n = 6) were removed and analyzed to determine the starting biofilm cell numbers via the drop dilution method. The 48-h old biofilms on the pegs were exposed to different concentrations of test antimicrobials, dissolved in an appropriate growth medium, for two h to evaluate their efficacy alone and in combination. Antimicrobial solutions tested against each organism included each agent alone at the MBEC, double combinations at the FBEC, and triple combinations ranging from 5 to 20% ethanol, 2.5-5 µg/mL chlorhexidine HCl and 1- 3% tetrasodium EDTA. Following treatment, pegs were washed twice with sterile PBS, and the biofilm cells were dislodged into recovery medium and enumerated as described above.

Antimicrobial activity of tetrasodium EDTA alone and in combination with either ethanol or chlorhexidine HCl against planktonic cells was determined. All three antimicrobials significantly inhibited the growth of all test organisms with MICs ranging from 0.063% to 2% for tetrasodium EDTA, 3.125%-12.5% for ethanol, and 0.1 µg/mL-50 µg/mL for chlorhexidine HCI. Synergy (FICI < 0.5) was detected with the combination of tetrasodium EDTA with ethanol for all test Gram-positive and fungal strains, whereas partial synergy (0.5 < FICI < 1.0) was observed for all Gram-negative strains. The combination of tetrasodium EDTA with chlorhexidine HCl showed indifferent activity (1 < FICI < 4) against 4 of 12 test strains and synergistic or partially synergistic activity against the eight remaining strains (Tables 2A-2C below).

TABLE 2A

Minimum Inhibitory Concentration of Tetrasodium EDTA, Ethanol, and Chlorhexidine HCl alone

| Organism | MIC | | |
|---|---|---|---|
| | EDTA (% w/v) | Ethanol (% w/v) | Chlorhexidine HCl (µg/mL) |
| Staphylococcus epidermidis ON170 | 0.063 | 3.125 | 0.1 |
| S. epidermidis SK9 | 0.063 | 6.25 | 0.1 |
| Staphylococcus aureus ON89 | 0.063 | 6.25 | 0.1 |
| MRSA ON184 | 0.063 | 6.25 | 0.2 |
| Stenotrophomonas maltophilia ON17 | 0.063 | 6.25 | 0.8 |
| Pseudomonas aeruginosa SK1 | 0.25 | 3.125 | 1.6 |
| Serratia marcescens SK2 | 2 | 6.25 | 12.5 |
| Proteus mirabilis ON153 | 1 | 6.25 | 50 |
| Escherichia coli ON29 | 0.5 | 12.5 | 0.4 |
| E. coli SK2 | 0.5 | 12.5 | 0.4 |
| Candida albicans SK4b | 1 | 6.25 | 1.6 |
| C. albicans ON47 | 1 | 6.25 | 1.6 |

TABLE 2B

Fractional Inhibitory Concentration Index (FICI) of Tetrasodium EDTA in Combination with Ethanol

| Organism | EDTA (% w/v) | Ethanol (% w/v) | FICl |
|---|---|---|---|
| Staphylococcus epidermidis ON170 | 0.015 | 0.4 | 0.36 (S) |
| S. epidermidis SK9 | 0.015 | 0.4 | 0.3 (S) |
| Staphylococcus aureus ON89 | 0.015 | 0.4 | 0.3 (S) |
| MRSA ON184 | 0.015 | 0.4 | 0.3 (S) |
| Stenotrophomonas maltophilia ON17 | 0.031 | 0.4 | 0.72 (PS) |
| Pseudomonas aeruginosa SK1 | 0.031 | 3.125 | 0.56 (PS) |
| Serratia marcescens SK2 | 0.5 | 3.125 | 0.75 (PS) |
| Proteus mirabilis ON153 | 0.5 | 1.6 | 0.75 (PS) |
| Escherichia coli ON29 | 0.25 | 3.125 | 0.75 (PS) |
| E. coli SK2 | 0.015 | 6.25 | 0.53 (PS) |
| Candida albicans SK4b | 0.25 | 1.6 | 0.5 (S) |
| C. albicans ON47 | 0.25 | 0.4 | 0.314 (S) |

TABLE 2C

Fractional Inhibitory Concentration Index (FICI) of Tetrasodium EDTA in Combination with Chlorhexidine HCl

| Organism | EDTA (% w/v) | Chlorhexidine HCl (µg/mL) | FICl |
|---|---|---|---|
| Staphylococcus epidermidis ON170 | 0.063 | 0.05 | 1.5 (I) |
| S. epidermidis SK9 | 0.015 | 0.025 | 0.5 (S) |
| Staphylococcus aureus ON89 | 0.008 | 0.1 | 1.126 (I) |
| MRSA ON184 | 0.008 | 0.2 | 1.126 (I) |
| Stenotrophomonas maltophilia ON17 | 0.015 | 0.2 | 0.5 (S) |
| Pseudomonas aeruginosa SK1 | 0.5 | 0.8 | 2.5 (I) |
| Serratia marcescens SK2 | 0.063 | 1.5 | 0.16 (S) |
| Proteus mirabilis ON153 | 0.063 | 0.8 | 0.08 (S) |
| Escherichia coli ON29 | 0.008 | 0.2 | 0.516 (PS) |
| E. coli SK2 | 0.008 | 0.1 | 0.266 (S) |
| Candida albicans SK4b | 0.063 | 0.8 | 0.596 (PS) |
| C. albicans ON47 | 0.125 | 0.8 | 0.658 (PS) |

The three antimicrobial agents displayed broad-spectrum microbicidal activity against the 12 test organisms. MBC or MFC values of all test antimicrobials were equal to or higher than their respective MICs. The combination of tetrasodium EDTA with either ethanol or chlorhexidine HCI showed synergistic and partially synergistic activity against all the test strains except S. epidermidis ON170, which showed additive activity with an FMCI of 1.0. The nature of interaction found in FICI was not always the same as the FMCI. However, none of the tested tetrasodium EDTA, ethanol, or chlorhexidine HCl combinations showed antagonism concerning the FICI and FMCI values. These results are shown in Tables 3A-3C.

TABLE 3A

Minimum Bactericidal Concentration (MBC) or Minimum Fungicidal Concentration (MFC) of Tetrasodium EDTA, Ethanol, and Chlorhexidine HCl alone

| Organism | MBC/MFC | | |
|---|---|---|---|
| | EDTA (% w/v) | Ethanol (% w/v) | Chlorhexidine HCl (µg/mL) |
| Staphylococcus epidermidis ON170 | 0.5 | 6.25 | 0.8 |
| S. epidermidis SK9 | 0.5 | 12.5 | 0.8 |
| Staphylococcus aureus ON89 | 1 | 25 | 0.8 |
| MRSA ON184 | 2 | 25 | 1.6 |
| Stenotrophomonas maltophilia ON17 | 1 | 6.25 | 3.125 |
| Pseudomonas aeruginosa SK1 | 1 | 12.5 | 3.125 |
| Serratia marcescens SK2 | 2 | 12.5 | 25 |
| Proteus mirabilis ON153 | 2 | 12.5 | 50 |
| Escherichia coli ON29 | 1 | 25 | 0.8 |
| E. coli SK2 | 0.5 | 25 | 0.8 |
| Candida albicans SK4b | 1 | 6.25 | 3.125 |
| C. albicans ON47 | 1 | 6.25 | 3.125 |

TABLE 3B

Fractional Microbicidal Concentration Index (FMCI) of Tetrasodium EDTA in Combination with Ethanol

| Organism | EDTA (% w/v) | Ethanol (% w/v) | FMCl |
|---|---|---|---|
| Staphylococcus epidermidis ON170 | 0.031 | 1.6 | 0.318 (S) |
| S. epidermidis SK9 | 0.031 | 3.125 | 0.312 (S) |
| Staphylococcus aureus ON89 | 0.25 | 3.125 | 0.375 (S) |
| MRSA ON184 | 0.031 | 3.125 | 0.14 (S) |
| Stenotrophomonas maltophilia ON17 | 0.125 | 1.6 | 0.381 (S) |
| Pseudomonas aeruginosa SK1 | 0.031 | 6.25 | 0.531 (PS) |
| Serratia marcescens SK2 | 0.25 | 6.25 | 0.625 (PS) |
| Proteus mirabilis ON153 | 0.063 | 6.25 | 0.53 (PS) |
| Escherichia coli ON29 | 0.25 | 6.25 | 0.5 (S) |
| E. coli SK2 | 0.25 | 6.25 | 0.75 (PS) |
| Candida albicans SK4b | 0.25 | 3.125 | 0.75 (PS) |
| C. albicans ON47 | 0.25 | 3.125 | 0.625 (PS) |

TABLE 3C

Fractional Microbicidal Concentration Index (FMCI) of Tetrasodium EDTA in Combination with Chlorhexidine HCl

| Organism | EDTA (% w/v) | Chlorhexidine HCl (µg/mL) | FMCl |
|---|---|---|---|
| Staphylococcus epidermidis ON170 | 0.25 | 0.4 | 1 (A) |
| S. epidermidis SK9 | 0.008 | 0.4 | 0.516 (PS) |
| Staphylococcus aureus ON89 | 0.008 | 0.2 | 0.375 (S) |
| MRSA ON184 | 0.125 | 0.4 | 0.312 (S) |
| Stenotrophomonas maltophilia ON17 | 0.031 | 0.4 | 0.16 (S) |
| Pseudomonas aeruginosa SK1 | 0.031 | 1.6 | 0.543 (PS) |
| Serratia marcescens SK2 | 0.125 | 1.6 | 0.126 (S) |
| Proteus mirabilis ON153 | 0.125 | 1.6 | 0.09 (S) |
| Escherichia coli ON29 | 0.008 | 0.2 | 0.258 (S) |
| E. coli SK2 | 0.015 | 0.2 | 0.258 (S) |
| Candida albicans SK4b | 0.125 | 0.8 | 0.381 (S) |
| C. albicans ON47 | 0.031 | 1.6 | 0.51 (PS) |

Compositions of tetrasodium EDTA alone and in combination with either ethanol or chlorhexidine HCI against 48-h old, preformed biofilms may be created using a single antimicrobial agent, effective at eradicating preformed biofilms of test pathogens, with concentrations between 4% to 0.0125% of tetrasodium EDTA, 100%-0.2% of ethanol, and 100 µg/mL-0.8 µg/mL of chlorhexidine HCI. As per CLSI guidelines, the MBEC is defined as the minimum concentration of an antimicrobial that eradicates 99.9% of micro-organisms (i.e., 3-log reduction) in a biofilm state compared with their respective growth controls in similar conditions. All antimicrobials achieved >99.99% (i.e., 4- log reduction) killing of bacterial biofilm cells, whereas the starting biofilm cell numbers for C. albicans were not enough to achieve a clinically recommended standard of biofilm killing.

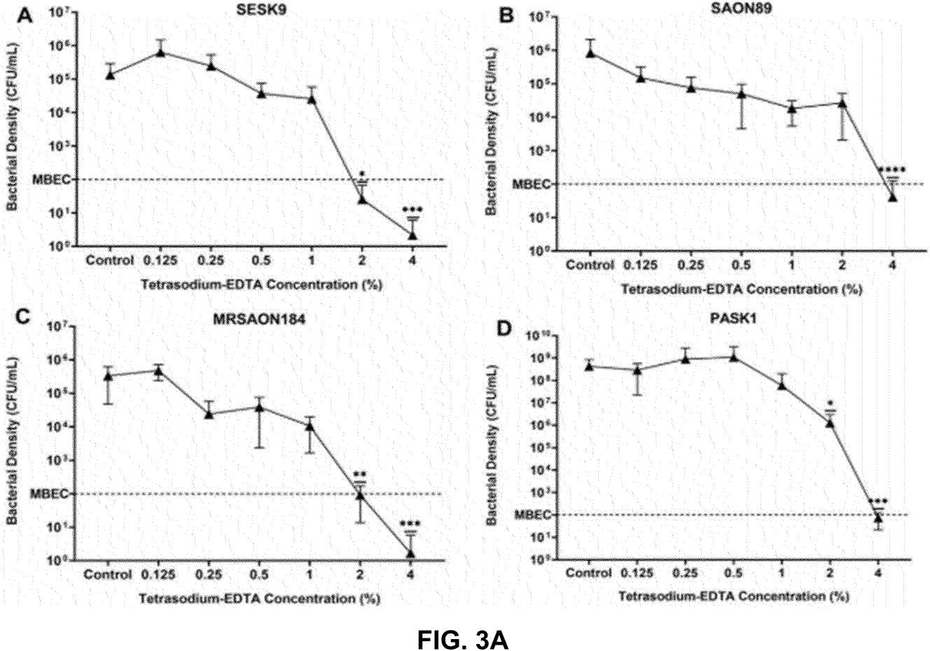

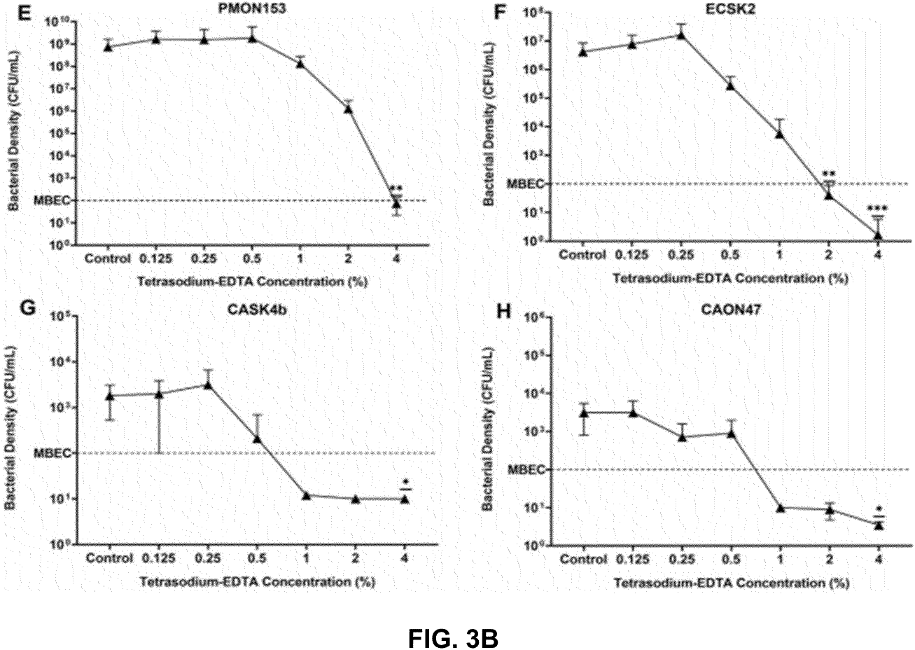

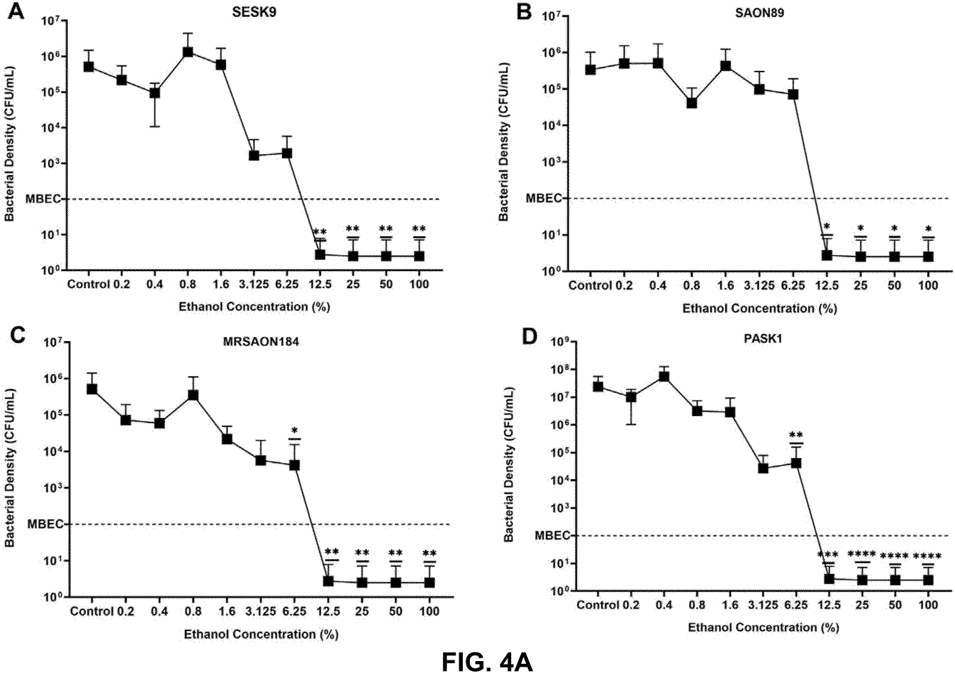

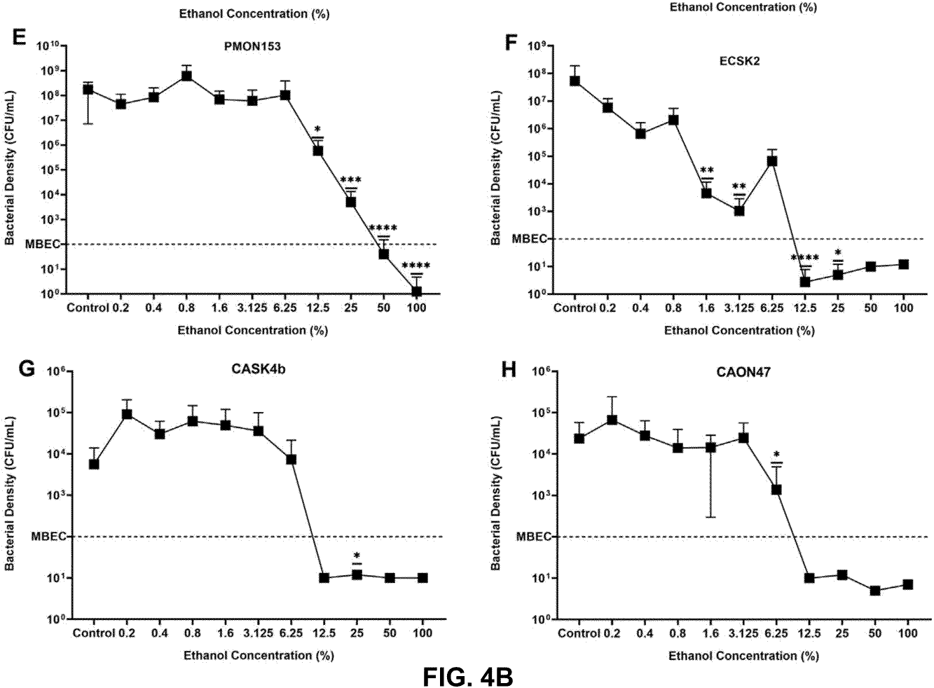

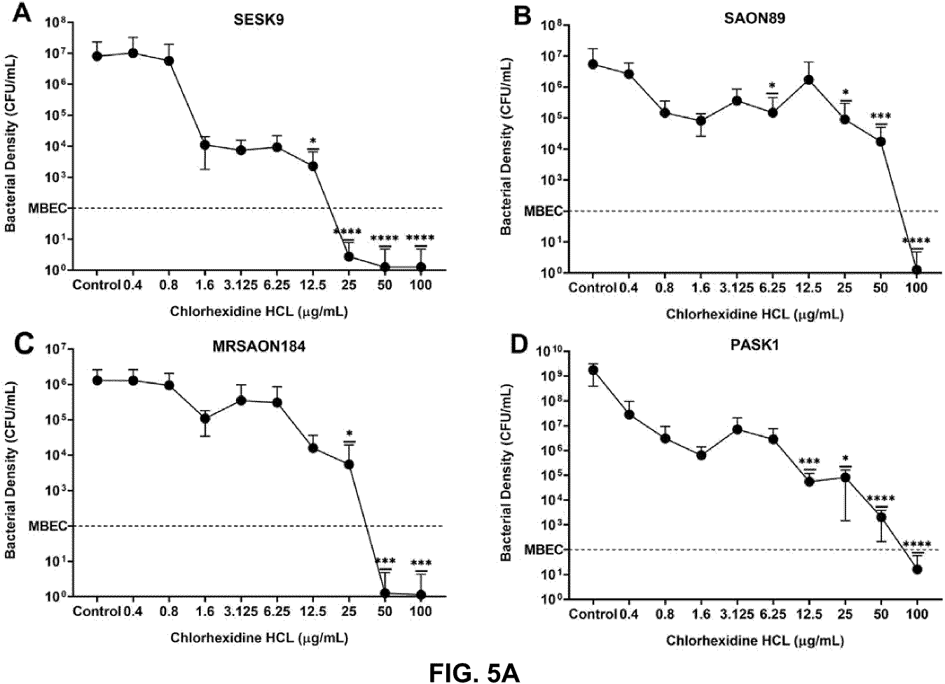

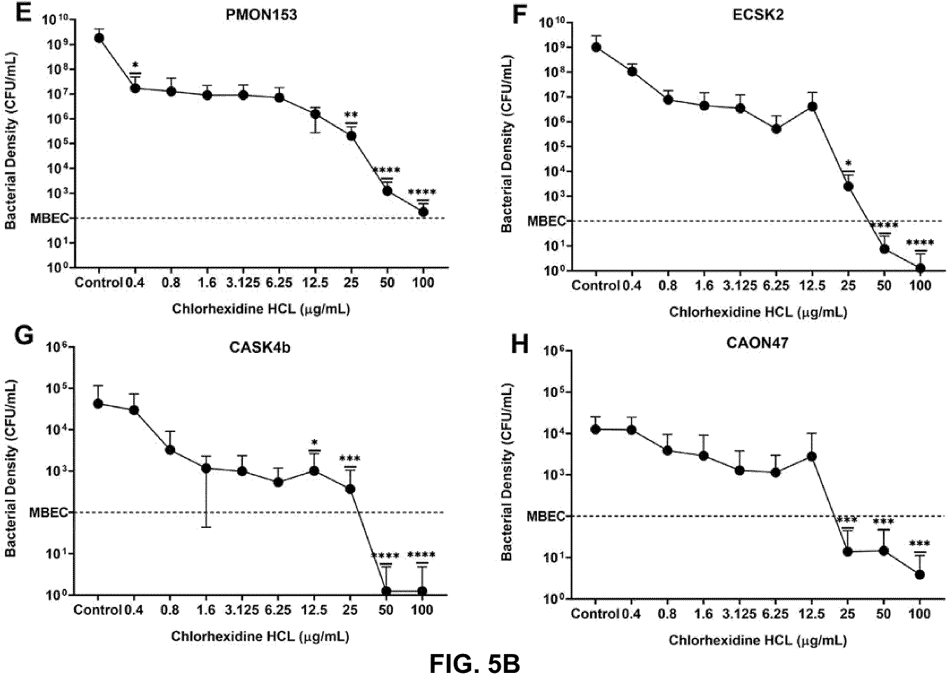

The MBEC of each antimicrobial agent against each test strain was established, and the data were plotted as the log reduction in the number of CFU (FIGS. 3A-5C). When tetrasodium EDTA was combined with either ethanol or chlorhexidine HCI, they exhibited a synergistic effect against all test strains in the study (Tables 4A-4C). According to the FBEC index, the concentration of tetrasodium EDTA in combination was decreased from ⅛- to 1/64-fold (with ethanol) and 1/16- to 1/64-fold (with chlorhexidine) in comparison with its original MBEC values. Also, the required concentrations dropped by ¼- to 1/16-fold for ethanol and ⅛- to 1/32-fold for chlorhexidine HCI when combined with tetrasodium EDTA (Tables 4A-4C).

TABLE 4A

Minimum Biofilm Eradication Concentration (MBEC) of Tetrasodium EDTA in Combination with Chlorhexidine HCI

| Organism | MBEC | | |
|---|---|---|---|
| | EDTA (% w/v) | Ethanol (% w/v) | Chlorhexidine HCI (µg/mL) |
| Staphylococcus epidermidis SK9 | 2 | 12.5 | 25 |
| Staphylococcus aureus ON89 | 4 | 12.5 | 100 |
| MRSA ON184 | 2 | 12.5 | 50 |
| Pseudomonas aeruginosa SK1 | 4 | 12.5 | 100 |
| Proteus mirabilis ON153 | 4 | 50 | 100 |
| Escherichia coli SK2 | 2 | 12.5 | 50 |
| Candida albicans SK4b | 1 | 12.5 | 50 |
| C. albicans ON47 | 1 | 12.5 | 25 |

TABLE 4B

Fractional Biofilm Eradication Concentration Index (FBECI) of Tetrasodium EDTA Combined with Ethanol

| Organism | EDTA (% w/v) | Ethanol (% w/v) | FBECI |
|---|---|---|---|
| Staphylococcus epidermidis SK9 | 0.031 | 1.6 | 0.14 (S) |
| Staphylococcus aureus ON89 | 0.063 | 0.8 | 0.08 (S) |
| MRSA ON184 | 0.063 | 1.6 | 0.16 (S) |
| Pseudomonas aeruginosa SK1 | 0.125 | 3.125 | 0.28 (S) |
| Proteus mirabilis ON153 | 0.125 | 6.25 | 0.16 (S) |
| Escherichia coli SK2 | 0.125 | 1.6 | 0.19 (S) |
| Candida albicans SK4b | 0.125 | 1.6 | 0.253 (S) |
| C. albicans ON47 | 0.125 | 1.6 | 0.253 (S) |

TABLE 4C

Fractional Biofilm Eradication Concentration Index (FBECI) of Tetrasodium EDTA Combined with Cholorhexidine HCI

| Organism | EDTA (% w/v) | Chlorhexidine HCl (µg/mL) | FBECl |
|---|---|---|---|
| Staphylococcus epidermidis SK9 | 0.125 | 3.125 | 0.19 (S) |
| Staphylococcus aureus ON89 | 0.125 | 3.125 | 0.06 (S) |
| MRSA ON184 | 0.031 | 6.25 | 0.14 (S) |
| Pseudomonas aeruginosa SK1 | 0.25 | 3.125 | 0.093 (S) |
| Proteus mirabilis ON153 | 0.125 | 3.125 | 0.06 (S) |
| Escherichia coli SK2 | 0.031 | 3.125 | 0.08 (S) |
| Candida albicans SK4b | 0.063 | 6.25 | 0.18 (S) |
| C. albicans ON47 | 0.031 | 6.25 | 0.28 (S) |

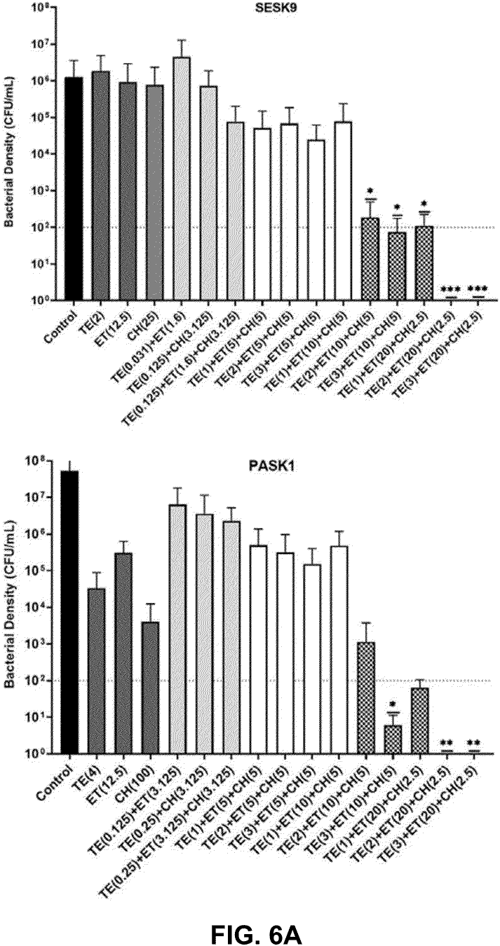

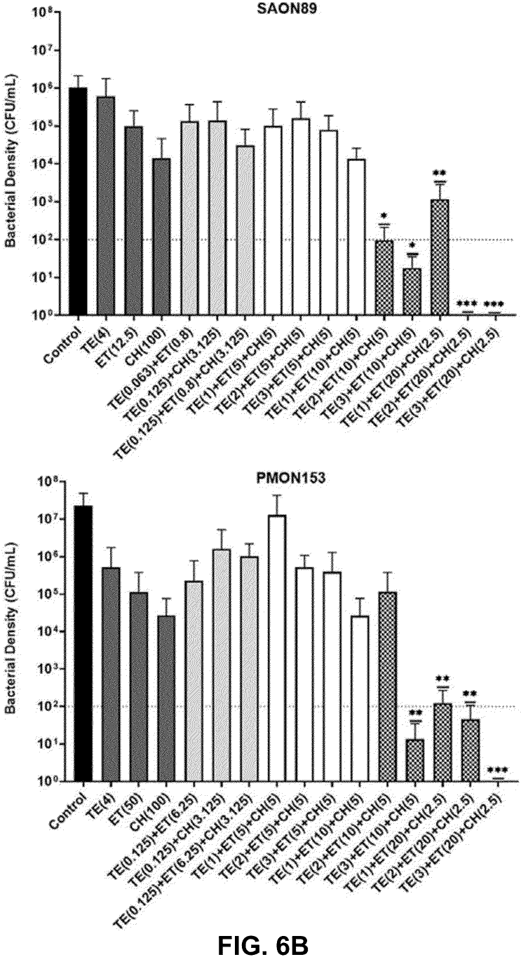

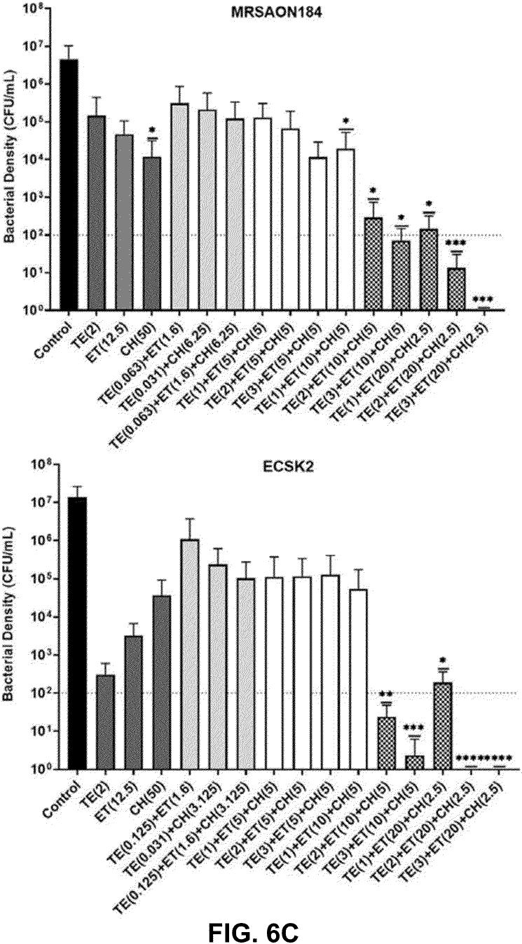

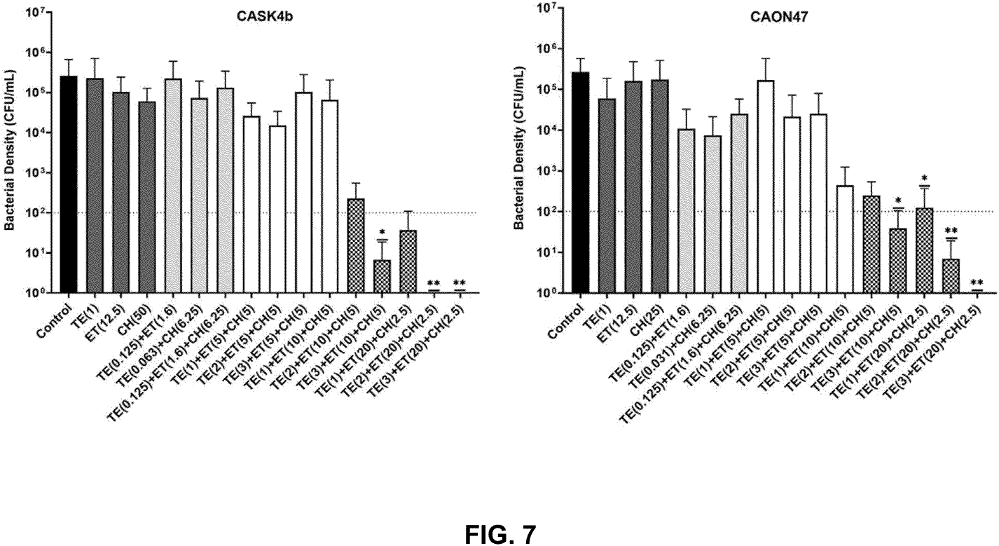

The method for rapid biofilm eradication ability of test antimicrobials alone and in combination against 48-h-old biofilms within two h exposure time included (1) choosing different concentrations of test antimicrobials to assess their potency in eradicating preformed biofilms of study organisms within two h. Then (2) the quantitative recovery from biofilms following exposure to the antimicrobial solutions for bacterial strains (FIGS. 6A-6C) and fungal strains (FIG. 7) was evaluated. Then (3) the exposure to the MBEC of tetrasodium EDTA, ethanol, or chlorhexidine HCI alone was measured as well as in several double and triple combinations of FBEC of antimicrobials failed to eradicate the preformed biofilms after two h of exposure. Then (4) all tested bacterial and fungal biofilms were entirely eradicated by the triple combination of 20% ethanol and 2.5 µg/mL chlorhexidine HCI in 3% tetrasodium EDTA (FIGS. 6A-7).

A triple combination of 20% ethanol and 2.5 µg/mL chlorhexidine HCl in 2% tetrasodium EDTA ultimately killed all biofilm cells except for three strains (MRSA ON184, P. mirabilis ON153, and C. albicans SK4b), but even for these strains, the viable cells were significantly reduced to at or near the limit of detection. Likewise, a combination of 1% tetrasodium EDTA with 20% ethanol and 2.5 µg/mL chlorhexidine HCI significantly reduced the viable cells in six of eight test organisms in comparison with their respective controls. A triple combination of 3% tetrasodium EDTA with 10% ethanol and five µg/mL chlorhexidine HCI also showed a significant reduction in viable biofilm cells of all test organisms within the 2-h contact time.

The results demonstrated that all test antimicrobials had efficient antimicrobial activity against planktonic and biofilm cells of test bacterial and fungal strains when exposed for 24 h. The combination of tetrasodium EDTA and ethanol was synergistic against planktonic cells of 6 of 12 strains tested, as measured by inhibition (FICI) and microbicidal (FMCI) activity. The interactions between tetrasodium EDTA and chlorhexidine HCI were categorized into synergistic, partially synergistic, additive, and indifferent activity against the test bacterial and fungal strains. It is noteworthy that there was no evidence of antagonistic activity between the three agents against planktonic cells in any tested combinations. We also tested the biofilm eradication ability of test antimicrobials against 48-h-old biofilms of bacterial and fungal strains within a 24-h exposure; 4% tetrasodium EDTA, 5% ethanol, and 100 µg/mL chlorhexidine HCl alone were able to eradicate all established biofilms following 24 h of treatment. As expected, biofilm cells were more resistant for each organism than planktonic cells. When tetrasodium EDTA was combined with ethanol or chlorhexidine HCl and used to treat biofilms, the agents worked synergistically, showing a remarkable reduction in concentrations compared with the MBEC values of single test antimicrobials. In many cases, the concentration of each agent required was near or lower than the MICs measured against planktonic cells. This strongly indicated that these three antimicrobials could be successfully used together to kill pathogenic microbes.

The combinations of antimicrobial agents showed efficient microbicidal activity against organisms within a reasonable contact time. Based on the results obtained from previous studies and the present study, concentrations of all three agents were chosen to optimize the effective combinations to eradicate biofilms within a selected 2-h exposure. The present study demonstrated that triple combinations of either 3% tetrasodium EDTA with 10% ethanol and 5 µg/mL chlorhexidine HCl or of 3% tetrasodium EDTA with 20% ethanol and 2.5 µg/mL chlorhexidine HCl completely eradicated 48-h-old biofilms of all of the test organisms following a 2-h exposure. In comparison with their individual antimicrobial effects, the combination of test antimicrobials significantly decreased the viable cells both of bacterial and fungal biofilms. The decrease in the ethanol concentration was compensated with an increased concentration of tetrasodium EDTA, and the effect was further accelerated with the addition of chlorhexidine HCl. The reduced ethanol concentration in the present study sets a more significant margin of safety from adverse reactions. In addition to improving safety, combination therapy may also decrease the risk of antimicrobial resistance among pathogens by reducing selection pressure. In addition, chlorhexidine concentrations above 2% have fewer human erythrocytes and neutrophils in vitro.

Additionally, toxicity of chlorhexidine is directly proportional to its concentration used. Considering this fact, the concentration of chlorhexidine HCl used in the triple combination was 0.00025% (w/v) in the present study.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present systems and methods, which, as a matter of language, might be said to fall therebetween.

What is claimed is:

1. A luer port cleaning cap device comprising:
a luer cap body affixable to a luer port, the luer cap body including an interior cavity; and
a seal enclosing a first composition within the interior cavity,
wherein the seal is configured to release the first composition a luer access device (LAD) comprising a luer port when the luer cap body is secured to the luer port of the LAD, and
wherein the first composition comprises EDTA having a concentration of at least about 1% (w/v).

2. The device of claim 1, wherein the first composition has a pH of at least 6.5.

3. The device of claim 1, wherein the first composition further comprises heparin, taurolidine, a thrombolytic agent, or a combination thereof.

4. The device of claim 1, wherein the first composition further comprises chlorhexidine or a pharmaceutically acceptable salt thereof.

5. The device of claim 4, wherein the chlorhexidine or a pharmaceutically acceptable salt thereof has a concentration in the composition from about 0.1 µg/mL to about 100 µg/mL.

6. The device of claim 1, wherein the first composition further comprises ethanol.

7. The device of claim 6, wherein the ethanol has a concentration in the first composition from about 0.1% (w/v) to about 70% (w/v).

8. The device of claim 3, wherein the first composition comprises heparin, and the heparin has a concentration in the first composition from about 1% (w/v) to about 8% (w/v).

9. The device of claim 3, wherein the first composition comprises a thrombolytic agent, and the thrombolytic agent comprises alteplase, streptokinase, reteplase, tenecteplase, urokinase, prourokinase, anistreplase, or a combination thereof.

10. The device of claim 9, wherein the thrombolytic agent comprises alteplase, urokinase, streptokinase, or a combination thereof.

11. The device of claim 9, wherein the thrombolytic agent has a concentration in the first composition of at least about 0.1% (w/v).

12. The device of claim 3, wherein the first composition comprises taurolidine, and the taurolidine has a concentration in the first composition from about 1% (w/v) to about 8% (w/v).

13. The device of claim 1, wherein the seal is disposed within the interior cavity.

14. The device of claim 1, wherein the luer cap body further comprises retention features.

15. The device of claim 1, wherein the luer cap body comprises a resilient thermoplastic material.

16. The device of claim 15, wherein the resilient thermoplastic material includes nylon, polycarbonate, polypropylene, or combinations thereof.

17. A luer connection cleaning cap kit comprising:
a luer cap including a first composition; and
a wipe packet including a towelette and a second composition,
wherein the first composition comprises EDTA at a concentration of at least about 1% (w/v), and
wherein the second composition comprises EDTA at a concentration of at least about 1% (w/v).

18. The kit of claim 17, wherein the towelette comprises an absorbent material capable of absorbing the second composition.

19. The kit of claim 17, wherein the first composition has a pH of at least 6.5.

20. The kit of claim 17, wherein the first composition further comprises heparin, taurolidine, a thrombolytic agent, or a combination thereof.

21. The kit of claim 17, wherein the first composition further comprises chlorhexidine or a pharmaceutically acceptable salt thereof.

22. The kit of claim 17, wherein the first composition further comprises ethanol.

23. The kit of claim 20, wherein the first composition comprises heparin, and the heparin has a concentration in the first composition from about 1% (w/v) to about 8% (w/v).

24. The kit of claim 20, wherein the first composition comprises a thrombolytic agent, and the thrombolytic agent comprises alteplase, streptokinase, reteplase, tenecteplase, urokinase, prourokinase, anistreplase, or a combination thereof.

25. The kit of claim 17, wherein first composition comprises taurolidine, and the taurolidine has a concentration in the first composition from about 1% (w/v) to about 8% (w/v).

26. The kit of claim 17, wherein the second composition further comprises heparin, taurolidine, a thrombolytic agent, or a combination thereof.

27. The kit of claim 17, wherein the second composition further comprises chlorhexidine or a pharmaceutically acceptable salt thereof.

28. The kit of claim 27, wherein the chlorhexidine or a pharmaceutically acceptable salt thereof has a concentration in the second composition from about 0.1 µg/mL to about 100 µg/mL.

29. The kit of claim 17, wherein the second composition further comprises ethanol.
